(19) United States
(12) Patent Application Publication
(10) Pub. No.: US 2016/0335400 A1
(43) Pub. Date: Nov. 17, 2016

(54) SYSTEMS AND METHODS FOR MANAGING PATIENT-CENTRIC DATA

(71) Applicant: Photon Medical Communications, Inc., Scottsdale, AZ (US)

(72) Inventors: Gregory Grant, Paradise Valley, AZ (US); John Vanderhoof, Scottsdale, AZ (US); Jason Allen Blood, Scottsdale, AZ (US)

(21) Appl. No.: 15/154,147

(22) Filed: May 13, 2016

Related U.S. Application Data
(60) Provisional application No. 62/160,934, filed on May 13, 2015.

(51) Int. Cl. G06F 19/00; H04L 29/06

(52) U.S. Cl. CPC G06F 19/322; G06F 19/345; G06F 19/3425; H04L 63/10; H04L 63/08

(57) ABSTRACT

System and methods are disclosed, one method comprises generating a patient profile comprising a patient identifier and information relating to the patient identifier, linking one or more service providers of the plurality of service providers to the generated patient profile, wherein the one or more linked service providers are capable of accessing the patient profile over a secure network connection, receiving an update relating to the patient profile, automatically transmitting a notice the one or more linked service providers, wherein the notice indicates that an update has been received relating to the patient profile, receiving a request to access the patient profile from the one or more linked service providers, and granting access to the patient profile including the update, via a secure network connection.

### CROSS-REFERENCE TO RELATED APPLICATIONS

This application claims priority under 35 U.S.C. §119 (e) to U.S. provisional application Ser. No. 62/160,934, filed on May 13, 2015, entitled “Systems and Methods for Managing Patient-Centric Data,” which is herein incorporated by reference in its entirety.

### BACKGROUND

Communication of diagnostic information and/or patient information can be tedious and time consuming. Currently, many physicians and care-providers rely on paging devices and voice calls/messages to communicate diagnostic information and consultation information to and from hospitals, offices, and other physicians, care providers and users of such information. For example, in an emergency room, a physician seeing a patient with an orthopedic problem has to page the orthopedic surgeon on call. The physician can often wait approximately 5-10 minutes or even longer for the orthopedic surgeon to respond. When the orthopedic surgeon finally responds, the orthopedic surgeon is typically provided only basic diagnostic information about the patient about which the emergency room physician is consulting. The orthopedic surgeon then has to find means to view certain patient diagnostic information, most notably images, such as x-rays. Computer terminals are not always conveniently available. Accordingly, the orthopedic surgeon must travel to a local hospital or office or another area to access a computer for viewing patient information and diagnostic information. Once the orthopedic surgeon has viewed the patient and diagnostic information, the orthopedic surgeon must communicate a response to the consulting physician in the emergency room. Typically, the orthopedic surgeon can rely on telephone communication to respond to the consulting physician with consulting information.

The current methods of consultation and remote diagnosis do not provide an efficient means of communicating diagnostic information and consultation responses to and from remote users. Furthermore, the current systems and methods do not provide a means to coordinate availability and schedules of recipients of consultation requests. These and other shortcomings are addressed by the present disclosure.

### SUMMARY

It is to be understood that both the following general description and the following detailed description are exemplary and explanatory only and are not restrictive, as claimed. In an aspect, provided are methods and systems for managing data and transmitting information. The system and methods of the present disclosure can be used to communication diagnostic information and response to and from remote users, such as physicians and care providers.

In an aspect, the systems and methods of the present disclosure minimize the communication inefficiencies and reduce the amount of wasted time that the emergency room physician and the patient have to deal with during their encounter. As an example, using the disclosed system and methods, an emergency room provider can determine that an orthopedist is necessary for consultation. The emergency room physician can chart like normal, but the data that is inputted into an electronic medical record (EMR) can be transmitted to a non-proprietary server, in which this information can be shared with any provider whether or not they have access to the proprietary system. The information inputted can be transmitted to the non-proprietary system with a single click of a mouse.

In an aspect, a discrete packet of information (e.g., Patient History Objective findings and Test results Over Network or PHOTON/Photon/photon) can be created once the emergency room provider requires consultation. As an example, the Photon can be sent to a cloud based server, which can be compatible with all or substantially all software platforms. The server can then push the Photon or packet of information to the remote physician or user, and the remote user is able to review the discrete packet of information with a user device, such as a smart device, tablet, computing device, or the like.

In an aspect, the remote user can review the information after logging-in in a secure manner. As an example, after reviewing the basic information and reviewing image information such as x-rays, the remote user can respond by transmitting a message (e.g., in a similar manner that the message was delivered) back to the original emergency room physician with diagnosis and disposition information. In an aspect, the emergency room physician can review the response provided by the remote user in a timely and secure manner.

In an aspect, a method can comprise receiving a request for information such as a diagnostic message. A selection of diagnostic information can also be received. At least a portion of the selected diagnostic information can be retrieved. The diagnostic message can be generated, wherein the diagnostic message comprises the portion of the selected diagnostic information. The diagnostic message can be transmitted to a recipient such as the requestor of information.

In an aspect, a method can comprise receiving a request for information such as a diagnostic message. Availability information relating to one or more of a plurality of users can be provided. A selection of at least one of the plurality of users can be received. A selection of diagnostic information can be received. Information relating to the selection of the at least one of the plurality of users and the selection of diagnostic information can be provided. The diagnostic message can be generated, wherein the diagnostic message comprises at least a portion of the selected diagnostic information. The diagnostic message can be transmitted to the at least one of the plurality of users selected.

In an aspect, a method can comprise receiving a first diagnostic message and receiving a second diagnostic message relating to the first diagnostic message. A plurality of response options can be provided, wherein at least one of the response options is customized based upon the first diagnostic message or the second diagnostic message, or both. A selection of one of the plurality of response options can be received. A responsive message can be provided based upon the selection of the one of the plurality of response options.

In an aspect, a method can comprise receiving a diagnostic message including diagnostic information. The diagnostic information can be rendered. A plurality of response options can be provided, wherein at least one of the response options is customized based upon the diagnostic information. A selection of one of the plurality of response options can be received. A message based upon the selection of the one of the plurality of response options can be generated and/or transmitted.

In another aspect, a system can comprise a memory for storing diagnostic information and a processor in communication with the memory. The processor can be configured to: receive a request for a diagnostic message; receive a selection of a first portion of the diagnostic information; retrieve a sub-portion of the selected first portion of the diagnostic information; generate the requested diagnostic message, wherein the diagnostic message comprises the sub-portion of the selected first portion of the diagnostic information; and provide the requested diagnostic message.

In another aspect, one method can comprise generating a patient profile comprising a patient identifier and information relating to the patient identifier, linking one or more service providers of the plurality of service providers to the generated patient profile, wherein the one or more linked service providers are capable of accessing the patient profile over a secure network connection, receiving an update relating to the patient profile, automatically transmitting a notice the one or more linked service providers, wherein the notice indicates that an update has been received relating to the patient profile, receiving a request to access the patient profile from the one or more linked service providers, and granting access to the patient profile including the update, via a secure network connection.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 is a block diagram of an exemplary network;

FIG. 2 is a block diagram of an exemplary computing device;

FIG. 3A is a flow chart of an exemplary method;

FIG. 3B is a flow chart of an exemplary method;

FIG. 3C is a flow chart of an exemplary method;

FIG. 4 is a flow chart of an exemplary method;

FIG. 5 is a representation of a user interface;

FIG. 6 is a representation of a user interface;

FIG. 7 is a representation of a user interface;

FIG. 8 is a representation of a user interface;

FIG. 9 is a representation of a user interface;

FIG. 10 is a representation of a user interface;

FIG. 11 is a representation of a user interface;

FIG. 12 is a representation of a user interface;

FIG. 13 is a representation of a user interface;

FIG. 14 is a representation of a user interface;

FIG. 15 is a representation of a user interface;

FIG. 16 is a representation of a user interface;

FIG. 17 is a representation of a user interface;

FIG. 18 is a representation of a user interface;

FIG. 19 is a representation of a user interface;

FIG. 20 is a representation of a user interface;

FIG. 21 is a representation of a user interface;

FIG. 22 is a representation of a user interface;

FIG. 23 is a representation of a user interface;

FIG. 24 is a representation of a user interface;

FIG. 25 is a representation of a user interface;

FIG. 26 is a representation of a user interface;

FIG. 27 is a representation of a user interface;

FIG. 28 is a flow diagram of an example method of operation;

FIG. 29 is a flow diagram of an example method of operation;

FIG. 30 is a flow diagram of an example method of operation;

FIG. 31 is a flow diagram of an example method of operation;

FIG. 32 is a screenshot of a user interface;

FIG. 33 is a screenshot of a user interface;

FIG. 34 is a screenshot of a user interface;

FIG. 35 is a screenshot of a user interface;

FIG. 36 is a screenshot of a user interface; and

FIG. 37 is a screenshot of a user interface.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification, PHOTON/Photon is an acronym which can stand for Patient History Objective findings and Test results Over Network. As used herein, PHOTON/Photon can comprise patient information, diagnostic information, medical images, and the like. In general, the Photon system provides a way to communicate a packet of patient information in a secure manner between healthcare professionals and, more importantly, allows communication regarding that information. The PHOTON packet can comprise a subset data relating to a patient, such as a small, highly relevant, customized set of data regarding a specific problem that a patient may be experiencing. The term PHOTON/Photon is used for example and illustration only. PHOTON/Photon is not intended to limit the underlying data represented thereby. Any data can be represented and is not limited by the term PHOTON/Photon to any particular definition or classification.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

The various features and processes described above may be used independently of one another, or may be combined in various ways. All possible combinations and sub-combinations are intended to fall within the scope of this disclosure. In addition, certain methods or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

It will also be appreciated that various items are illustrated as being stored in memory or on storage while being used, and that these items or portions thereof may be transferred between memory and other storage devices for purposes of memory management and data integrity. Alternatively, in other embodiments some or all of the software modules and/or systems may execute in memory on another device and communicate with the illustrated computing systems via inter-computer communication. Furthermore, in some embodiments, some or all of the systems and/or modules may be implemented or provided in other ways, such as at least partially in firmware and/or hardware, including, but not limited to, one or more application-specific integrated circuits (“ASICs”), standard integrated circuits, controllers (e.g., by executing appropriate instructions, and including microcontrollers and/or embedded controllers), field-programmable gate arrays (“FPGAs”), complex programmable logic devices (“CPLDs”), etc. Some or all of the modules, systems and data structures may also be stored (e.g., as software instructions or structured data) on a computer-readable medium, such as a hard disk, a memory, a network, or a portable media article to be read by an appropriate device or via an appropriate connection. The systems, modules and data structures may also be transmitted as generated data signals (e.g., as part of a carrier wave or other analog or digital propagated signal) on a variety of computer-readable transmission media, including wireless-based and wired/cable-based media, and may take a variety of forms (e.g., as part of a single or multiplexed analog signal, or as multiple discrete digital packets or frames). Such computer program products may also take other forms in other embodiments. Accordingly, the present invention may be practiced with other computer system configurations.

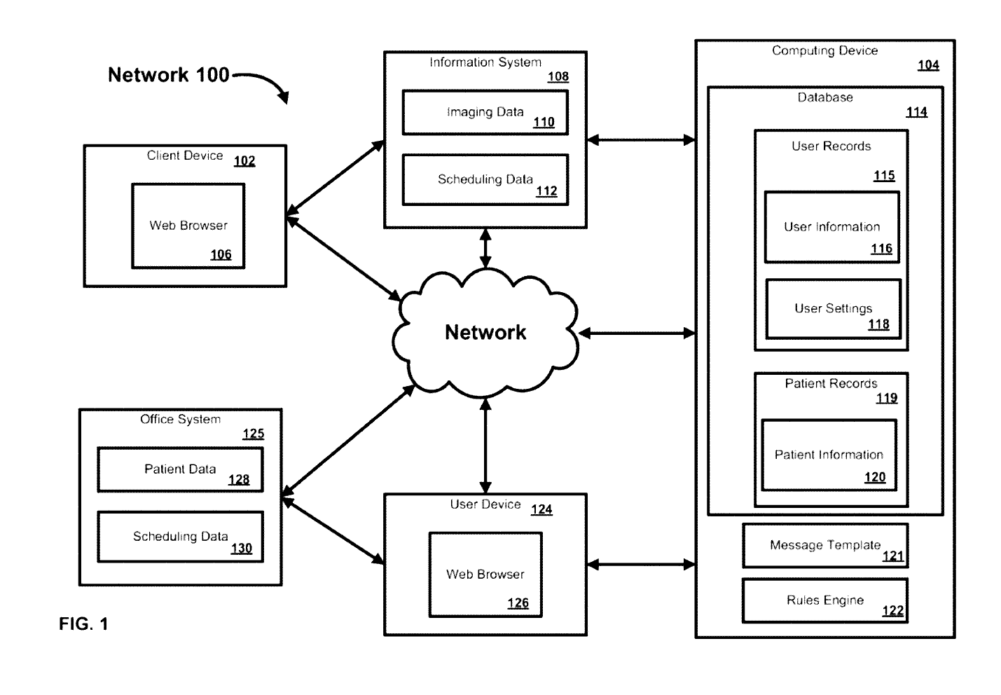

FIG. 1 illustrates various aspects of an exemplary network in which the present methods and systems can operate. The present disclosure relates to systems and methods for managing data such as medical related information. Those skilled in the art will appreciate that present methods may be used in systems that employ both digital and analog equipment. One skilled in the art will appreciate that provided herein is a functional description and that the respective functions can be performed by software, hardware, or a combination of software and hardware.

The system and network 100 can comprise a client device 102 in communication (e.g., directly and/or via a network) with a computing device 104 such as a server, for example. The computing device 104 can be disposed locally or remotely relative to the client device 102. As an example, the client device 102 and the computing device 104 can be in communication via a private or public network such as the Internet. Other forms of communications can be used such as wired and wireless telecommunication channels, for example.

In an aspect, the client device 102 can be an electronic device such as a computer, a server, a smartphone, a laptop, a tablet, or other device capable of communicating with the computing device 104. As an example, the client device 102 can comprise a web browser 106 for providing an interface to a user to interact with the client device 102 and/or the computing device 104. The web browser 106 can be any interface for presenting information to the user and receiving a user feedback, such as Internet Explorer, Mozilla Firefox, Google Chrome, Safari, or the like. Other software, hardware, and/or interfaces can be used to provide communication between the user and one or more of the client device 102 and the computing device 104. As an example, the web browser 106 can request or query various files from a local source and/or a remote source. As a further example, the client device 102 can be configured to transmit data to the computing device 104. Other devices and interfaces can be used to allow a user to intercommunicate with the computing device 104. In an aspect, the client device 102 can be authenticated via user/device credentials prior to communicating secure information to the computing deice 104 or other device. As an example, the client device 102 can comprise software to facilitate secure communication and/or authentication.

In an aspect, the client device 102 can be configured to communicate (e.g., directly and/or via a network) with an information system 108 (e.g. emergency room information system, hospital information system, imaging system, scheduling system, etc.). As an example, the information system 108 can comprise a database. As a further example, the information system 108 can comprise hardware (e.g., terminal) and or software components for storing and/or processing patient information such as imagining data 110 (e.g., x-rays, CT scans, EKGs, etc.). Other information can be stored and/or processed by the information system 108, such as scheduling data 112 (e.g., relating to “on-call” physicians, employee schedules, or other schedule and time related data). In an aspect, the client device 102 can send/receive information to/from the information system 108 for storing information in the information system 108 and/or retrieving information from the information system 108. As an example, the client device 102 can comprise an instruction set or rule set to control the transmission of data to/from the information system 108. As a further example, the client device 102 can be configured to push data to the information system 108 when an input data is received by the client device 102. Any data or portion of data inputted to the client device 102 can be selectively and/or automatically transmitted to the information system 108 or another storage medium. In an aspect, the client device 102 can be authenticated via user/device credentials prior to communicating secure information to the information system 108 or other device. As an example, the client device 102 can comprise software to facilitate secure communication and/or authentication.

In an aspect, the information system 108 can comprise data relating to a hospital, clinic, medical facility, emergency room, or other professional environment. Other data can be stored and processed by the information system 108. As an example, the information system 108 can be located remotely from the client device 102. As a further example, the information system 108 can be integrated with the client device 102 or in communication with the client device over a local network.

In an aspect, the computing device 104 can be a server for communicating with the client device 102. As an example, the computing device 104 can be configured to receive message requests (e.g., diagnostic messages) from another device, such as the client device 102. The computing device 104 can be configured to process the message request and generate/transmit a message in response to the request. As a further example, the computing device 104 can manage the intercommunication between the client device 102 and a database 114 for sending and receiving data therebetween. In an aspect, the database 114 can store a plurality of files (e.g. web pages). As an example, the client device 102 can request a file from the database 114. As a further example, the client device 102 can retrieve a file from the database 114.

In an aspect, the database 114 can store a plurality of user records 115. As an example, one or more of the user records 115 can comprise user information 116 relating to a client or other user. In an aspect, the user information 116 can comprise contact information, professional/license information, preferences, and mailing lists, for example.

In an aspect, one or more user records 115 can comprise user settings 118 relating to one or more users, physicians, consultants, healthcare providers, professionals, message recipients, and the like. As an example, the user settings 118 can comprise demographic information, contact information, user credentials or login credentials, a unique identifier or password, and preferences (e.g. message preferences, including pre-defined information fields to be populated and included in diagnostic messages).

In an aspect, the database 114 can store a plurality of patient records 119. As an example, one or more of the patient records 119 can comprise patient information 120 relating to a client or other user. In an aspect, the patient information 120 can comprise contact information, medical information, insurance information, preferences, and other information relating to patient and/or treatment, for example. Other information can be stored in the database 114 and/or associated with a particular patient record 119.

In an aspect, one or more message templates 121 can be retrieved by the computing device 104 (e.g., stored in the database 114 or in other storage devices/media). As an example one or more message templates 121 can comprise a pre-defined layout of a plurality of information fields. As a further example, each of the message templates 121 can comprise a plurality of information fields. In an aspect, the information fields of the message templates can be populated from data stored in the database 114. However, the information fields of the message templates 121 can be populated from any data source. Any number of information fields representing any data or information can be included in the message templates 121. Any number of message templates 121 can be stored and/or generated. As an example, one or more message templates 121 can comprises one or more data fields relating to one or more of the following: demographics, such as first name, middle name, last name, date of birth, SSN, address, driver's license information, insurance information, second insurance information, health information; chief complaint (e.g., what is patient is primarily complaining about?); history of present illness (e.g. what happened to the patient? how did it happen? where did it happen? associated symptoms, pain level, etc.); past medical history; past surgical history; medications; allergies; social history (e.g., history of smoking, drinking, drugs. employment, living situation); review of systems (e.g. history of fever, headache, chest pain, shortness of breath; physical examination information; general appearance (e.g., what the patient looks like—thin, obese, disheveled, clean, etc.); mood and affect; psychiatric information; neurologic exam; integumentary exam (skin); cardiovascular exam (blood vessels); studies, including labs such as complete blood count, blood electrolytes, liver function tests, urinalysis, drug screen, alcohol level, arterial blood gas, cardiac enzymes, troponin, erythrocyte sedimentation rate, C-reactive protein; imaging including X-rays, MRI, CT, ultrasound, echocardiogram, bone scan, PET scan, EKG; audio/video, clinical photographs; impression/diagnosis, such as triage nurse impression, physician assistant impression, ED physician impression; ED Action Plan; and/or disposition and follow-up appointment.

In an aspect, the message can comprise complete patient encounter information. The users that will receive the diagnostic message can have the option of selecting a discrete amount of highly relevant information for their particular specialty. Other users can choose to accept all the information. Each specialty will be different from each other with regards to what information is most relevant.

In an aspect, the computing device 104 can comprise a rules engine 122 for applying one or more rules/filter/instructions/settings to the messages (e.g., diagnostic messages). As an example, the rules engine 122 can retrieve preferences and instructions from the user settings 118 in order to customize a particular message format and/or content associated with a particular recipient. In this way, a recipient can define a particular information and formation of the information to be included in any messages received by the particular recipient. As a further example, certain information is customizable and other information and formatting is standard. In an aspect the rules engine 122 can customize format/content based upon any number of rules or instructions, such as information about the sender or recipient, information specific to the client or patient/subject of the message, specialty, time of day, means of communication, level of urgency, and other rules.

In an aspect, a user device 124 can be in communication with the computing device 104. The computing device 104 can be disposed locally or remotely relative to the user device 124. As an example, the user device 124 and the computing device 104 can be in communication via a private or public network, such as the Internet. Other forms of communications can be used such as wired and wireless telecommunication channels, for example.

In an aspect, the user device 124 can be an electronic device, such as a computer, a server, a smartphone, a laptop, a tablet, or other device capable of communicating with the computing device 104. As an example, the user device 124 can comprise a communication element, such as web browser 126 for providing an interface to a user to interact with the client device 102, the computing device 104, the information system 108, and/or an office system 125. The web browser 126 can be any interface for presenting information to the user and receiving a user feedback, such as Internet Explorer, Mozilla Firefox, Google Chrome, Safari, or the like. Other software, hardware, and/or interfaces can be used to provide communication between the user and one or more of the user device 124 and the client device 102, the computing device 104, the information system 108, and/or an office system 125. As an example, the web browser 126 can request or query various files from a local source and/or a remote source. As a further example, the user device 124 can be configured to transmit data to the computing device 104 via various protocols and over various networks. Other devices and interfaces can be used to allow a user to intercommunicate with the computing device 104. In an aspect, the client device 102 can be authenticated via user/device credentials prior to communicating secure information to the client device 102, the computing device 104, the information system 108, and/or an office system 125, or other device. As an example, the client device 102 can comprise software to facilitate secure communication and/or authentication. In an aspect, a user can use the user device 124 to communicate with client device 102 to transmit/receive data therebetween. As an example, the client device 102 can operate as a proxy for the user device 104 when communicating with the computing device 104, the information system 108, and/or an office system 125. As a further example, the user device 124 may not be authenticated with one or more of the computing device 104, the information system 108, and/or an office system 125. Accordingly, the user device 124 can communicate information to the client device 102 using a unique identifier (e.g., temporary or persistent), and the client device 102 can communication with one or more of the computing device 104, the information system 108, and/or an office system 125 on behalf of the user device 124.

In an aspect, the office system 125 can comprise information relating to a particular professional office, such as a medical office, law office, or other group of professionals. As an example, the office system 125 can be located remote from the information system 108 and/or the computing system 104. In an aspect, the office system 125 can comprise a patient data 128 (e.g., EMR) and/or a scheduling data 130. As an example, the patient data 128 can relate to a client or other user. In an aspect, patient data 128 can comprise contact information, medical information, insurance information, preferences, and other information relating to patient and/or treatment, for example. Other information can be stored in office system 125 and/or associated with a particular user/patient. As a further example, the scheduling data 130 can comprise information relating to a schedule of one or more physicians, healthcare providers, staff, technicians, professionals, or other office personnel. In an aspect, the client device 102 can be authenticated via user/device credentials prior to communicating secure information to the office system 125 or other device. As an example, the client device 102 can comprise software to facilitate secure communication and/or authentication.

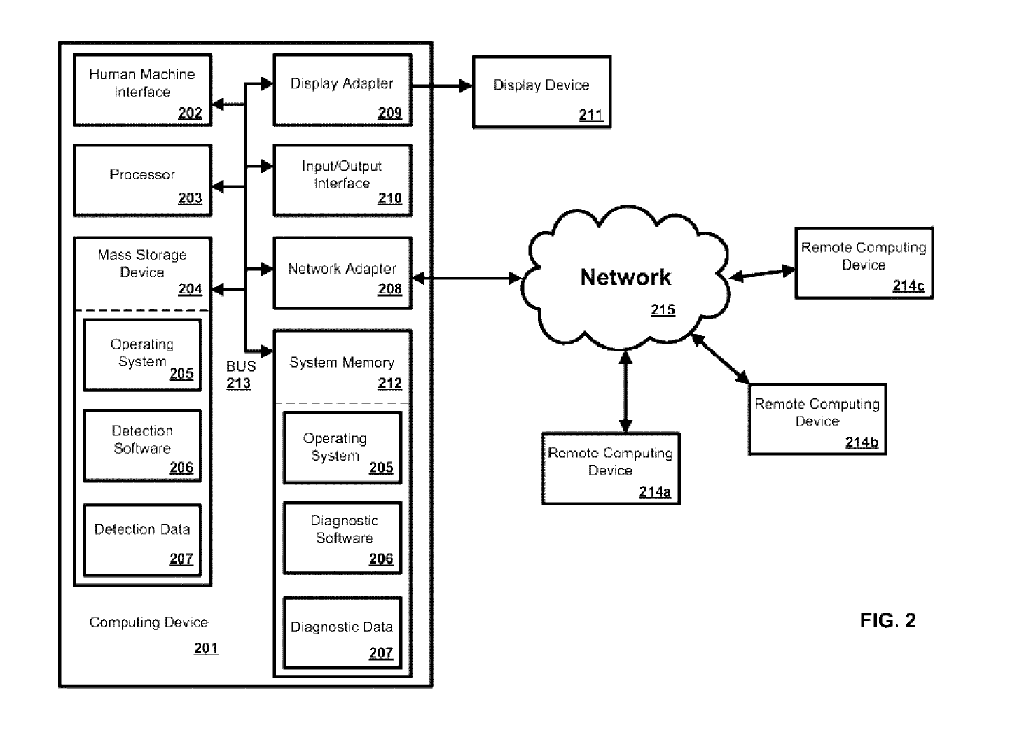

In an exemplary aspect, the methods and systems can be implemented on a computing system such as computing device 201 as illustrated in FIG. 2 and described below. By way of example, one or more of the client device 102, the computing device 104, and the user device 124 of FIG. 1 can be a computer as illustrated in FIG. 2. Similarly, the methods and systems disclosed can utilize one or more computers to perform one or more functions in one or more locations. FIG. 2 is a block diagram illustrating an exemplary operating environment for performing the disclosed methods. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The present methods and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

FIG. 2 is a block diagram illustrating an exemplary operating environment for performing the disclosed methods. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The present methods and systems can be operational with numerous other general purpose or special purpose computing system environments or configurations. Examples of well known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods disclosed herein can be implemented via a general-purpose computing device in the form of a computer 201. The components of the computer 201 can comprise, but are not limited to, one or more processors or processing units 203, a system memory 212, and a system bus 213 that couples various system components including the processor 203 to the system memory 212. In the case of multiple processing units 203, the system can utilize parallel computing.

The system bus 213 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 213, and all buses specified in this description can also be implemented over a wired or wireless network connection and each of the subsystems, including the processor 203, a mass storage device 204, an operating system 205, financial software 206, financial data 207, a network adapter 208, system memory 212, an Input/Output Interface 210, a display adapter 209, a display device 211, and a human machine interface 202, can be contained within one or more remote computing devices 214a,b,c at physically separate locations, connected through buses of this form, in effect implementing a fully distributed system.

The computer 201 typically comprises a variety of computer readable media. Exemplary readable media can be any available media that is accessible by the computer 201 and comprises, for example and not meant to be limiting, both volatile and non-volatile media, removable and non-removable media. The system memory 212 comprises computer readable media in the form of volatile memory, such as random access memory (RAM), and/or non-volatile memory, such as read only memory (ROM). The system memory 212 typically contains data such as financial data 207 and/or program modules such as operating system 205 and financial software 206 that are immediately accessible to and/or are presently operated on by the processing unit 203.

In another aspect, the computer 201 can also comprise other removable/non-removable, volatile/non-volatile computer storage media. By way of example, FIG. 2 illustrates a mass storage device 204 which can provide non-volatile storage of computer code, computer readable instructions, data structures, program modules, and other data for the computer 201. For example and not meant to be limiting, a mass storage device 204 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 204, including by way of example, an operating system 205 and financial software 206. Each of the operating system 205 and financial software 206 (or some combination thereof) can comprise elements of the programming and the financial software 206. Financial data 207 can also be stored on the mass storage device 204. Financial data 207 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computer 201 via an input device (not shown). Examples of such input devices comprise, but are not limited to, a keyboard, pointing device (e.g., a “mouse”), a microphone, a joystick, a scanner, tactile input devices such as gloves, and other body coverings, and the like These and other input devices can be connected to the processing unit 203 via a human machine interface 202 that is coupled to the system bus 213, but can be connected by other interface and bus structures, such as a parallel port, game port, an IEEE 1394 Port (also known as a Firewire port), a serial port, or a universal serial bus (USB).

In yet another aspect, a display device 211 can also be connected to the system bus 213 via an interface, such as a display adapter 209. It is contemplated that the computer 201 can have more than one display adapter 209 and the computer 201 can have more than one display device 211. For example, a display device can be a monitor, an LCD (Liquid Crystal Display), or a projector. In addition to the display device 211, other output peripheral devices can comprise components such as speakers (not shown) and a printer (not shown) which can be connected to the computer 201 via Input/Output Interface 210. Any step and/or result of the methods can be output in any form to an output device. Such output can be any form of visual representation, including, but not limited to, textual, graphical, animation, audio, tactile, and the like.

The computer 201 can operate in a networked environment using logical connections to one or more remote computing devices 214a,b,c. By way of example, a remote computing device can be a personal computer, portable computer, a server, a router, a network computer, a peer device or other common network node, and so on. Logical connections between the computer 201 and a remote computing device 214a,b,c can be made via a local area network (LAN) and a general wide area network (WAN). Such network connections can be through a network adapter 208. A network adapter 208 can be implemented in both wired and wireless environments. Such networking environments are conventional and commonplace in offices, enterprise-wide computer networks, intranets, and the Internet 215.

For purposes of illustration, application programs and other executable program components such as the operating system 205 are illustrated herein as discrete blocks, although it is recognized that such programs and components reside at various times in different storage components of the computing device 201, and are executed by the data processor(s) of the computer. An implementation of financial software 206 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems can employ artificial intelligence (AI) techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

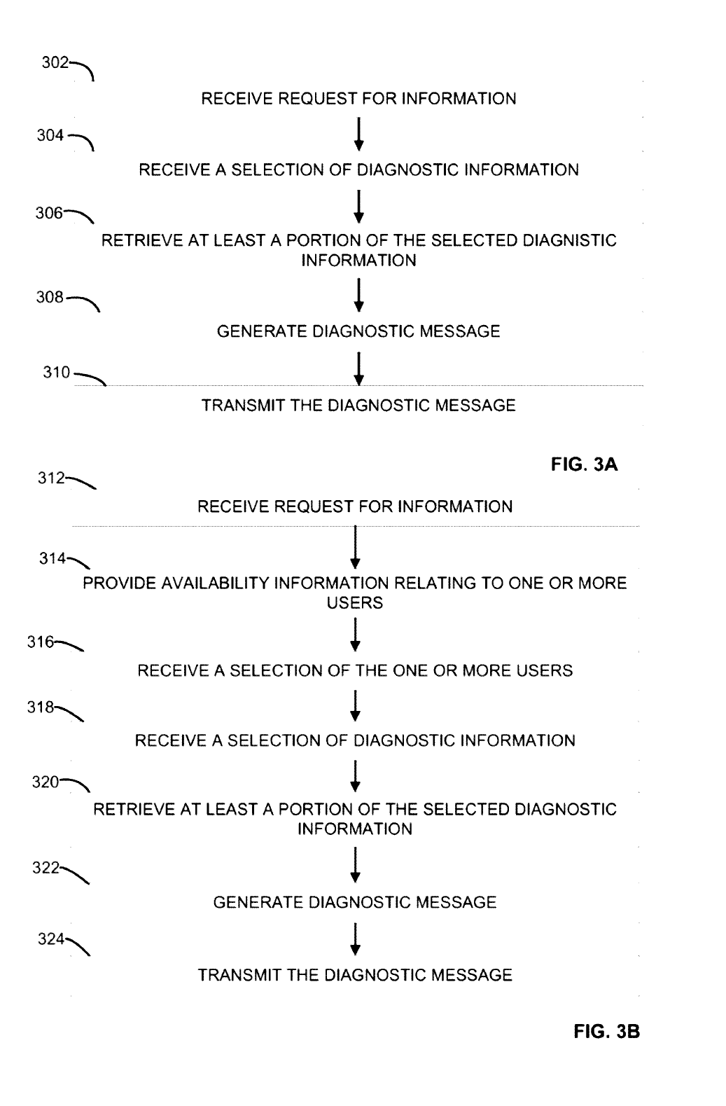

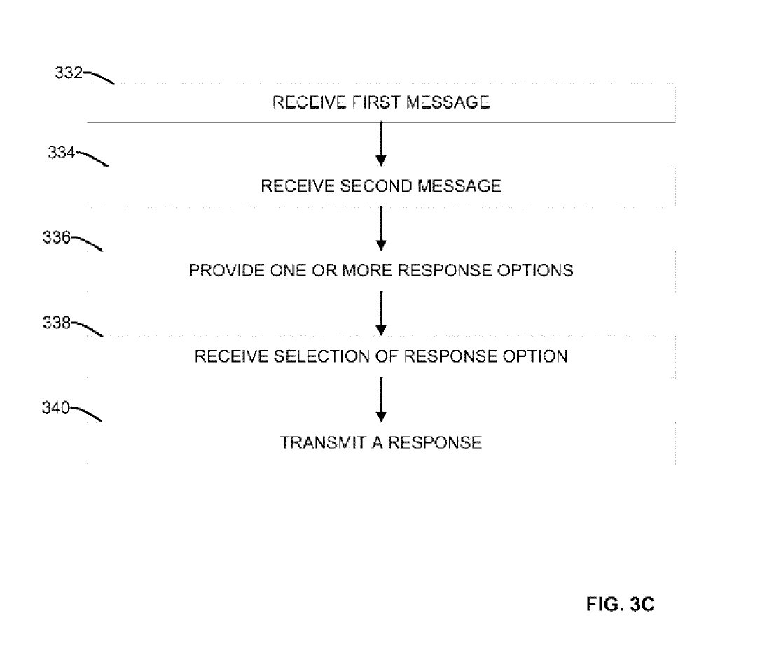

In an aspect, FIG. 3A illustrates an exemplary method for generating and/or transmitting a message such as a diagnostic message or consultation message. In an aspect, in step 302, a request for information such as a message (e.g., diagnostic message, PHOTON, or the like) can be received, for example, by the computing device 104. As an example, the request can be received by the client device 102 or the user device 124. However, other devices and systems can be used to transmit the request. In an aspect, an interface is provided to allow a user/client to request a message. As an example, the client device 102 can include a user interface with a “request message” icon or engageable button. Other means of requesting the message can be used. As a further example, once a user/client selects the “request message” icon, the user/client can be prompted to provide and/or select particular diagnostic information to be included in the message.

In an aspect, the client can select/provide a particular information (e.g., diagnostic information, requester information, recipient information, etc.) to be included in the message and can submit the request for the message. In an aspect, in step 304, the selection/provision of information can be received, for example, by the computing device 104. As a further example, the computing device 104 can process the request to retrieve at least a portion of the selected information (e.g., diagnostic information), at step 306. In an aspect, the diagnostic information can comprises one or more of patient data, medical data, and a medical image such as an EKG, an X-ray and/or other medical images, audio or video.

In an aspect, the computing device 104 can retrieve the selected/requested diagnostic information from any storage medium(s). As an example, at least a portion of the selected/requested diagnostic information can be retrieved from the database 114. In an aspect, the user settings 118 associated with an intended recipient of the diagnostic message can be applied to the selected/requested diagnostic information. For example, a particular user setting 118 may include pre-defined information fields that should be included in the messages that are sent to the user/recipient associated with the particular user setting 118. Accordingly, even though the selected/requested diagnostic information includes different information, the user setting 118 can take priority to retrieve a subset of the selected/requested diagnostic information to be included in the message.

In an aspect, in step 308, a diagnostic message can be generated. As an example, the diagnostic message can comprise the portion of the selected diagnostic information. However, any information can be included. In an aspect, the user settings 118 associated with an intended recipient of the diagnostic message are applied to the selected/requested diagnostic information. For example, a particular user setting 118 may include pre-defined information fields to be included in the messages that are sent to the user/recipient associated with the particular user setting 118. Accordingly, even though the selected/requested diagnostic information includes different information, the user setting 118 can take priority to retrieve a subset of the selected/requested diagnostic information to be included in the message.

In an aspect, computing device 104 and/or database 114 can comprise specialty templates for the messages. The specialty templates can be specifically created for each specialty in a particular profession, such as medicine/healthcare. For example, an orthopedic template can consist of basic patient information as well as x-rays. As a further example, a cardiology template can consist of basic patient information as well as an EKG or echocardiogram. As a further example, a gastroenterology template will consist of basic information as well as an abdominal x-ray or abdominal CT and perhaps lab information. The specialty templates can be created prior to implementation of the specific specialty message generation. Templates can be changed as needed. In an aspect, the requester of a message can select a pre-defined template for sending to an intended recipient.

In step 310, the message (e.g., diagnostic message) can be made available to one or more users (e.g., accessible via a communication link). As an example, the message can be made available to a recipient identified by the requester of the message. As another example, the message can be made available (e.g., transmitted) over any communication path or network such as the Internet and/or mobile telephone network.

In an aspect, the computing device 104 or other device can track a status of one or more transmitted messages. As an example, the following information can be tracked, time stamped, and or stored: date/time the message was generated/sent/received/forwarded/replied to; location (GPS) of requester/recipient; recipient device; receipt confirmation; read confirmation; no-read alert/feedback; and returned message alert. Other information and a feedback can be tracked/stored. In an aspect, the tracked information can be used to document the timeline of the electronic consultation and provide some medicolegal cushion for the emergency room providers who have to rely strictly on a verbal order or recommendation from the consultants. As an example, consultants who render medical decisions verbally can now be formally documented in the medical record when they made their diagnosis and what kind of treatment was recommended. This provides an enormous amount of medicolegal relief to ED providers.

FIG. 3B illustrates an exemplary method for managing a diagnostic data based upon recipient availability. In an aspect, in step 312, a request for information, such as a message (e.g., diagnostic message, PHOTON, or the like), can be received, for example, by the computing device 104. As an example, the request can be received by the client device 102 or the user device 124. However, other devices and systems can be used to transmit the request. In an aspect, an interface is provided to allow a user/client to request a message. As an example, the client device 102 can include a user interface with a “request message” icon or engageable button. Other means of requesting the message can be used. As a further example, once a user/client selects the “request message” icon, the user/client can be prompted to provide and/or select particular diagnostic information to be included in the message.

In an aspect, in step 314, availability information relating to one or more of a plurality of users or intended recipients can be rendered to the client or requester. As an example, a calendar of availability for one or more on-call professionals, such as healthcare providers, can be rendered to the client or requester.

In an aspect, in step 316, a selection of at least one of the plurality of users or intended recipients can be received. In an aspect, the client selects/provides a desired recipient of the message based upon the availability information. As an example, the client can select an available recipient from a list of one or more on-call users. In an aspect, the selection/provision of availability information is received, for example, by the computing device 104.

In an aspect, the client selects/provides a particular information (e.g., diagnostic information, requester information, recipient information, etc.) to be included in the message and submits the request for the message. In an aspect, in step 318, the selection/provision of information is received, for example, by the computing device 104. As a further example, the computing device 104 can process the request to retrieve at least a portion of the selected information (e.g., diagnostic information), at step 320. In an aspect the diagnostic information can comprises one or more of patient data, medical data, and a medical image.

In an aspect, the computing device 104 can retrieve the selected/requested diagnostic information from any storage medium(s). As an example, at least a portion of the selected/requested diagnostic information is retrieved from the database 114. In an aspect, the user settings 118 associated with an intended recipient of the diagnostic message are applied to the selected/requested diagnostic information. For example, a particular user setting 118 may include pre-defined information fields that should be included in the messages that are sent to the user/recipient associated with the particular user setting 118. Accordingly, even though the selected/requested diagnostic information includes different information, the user setting 118 can take priority to retrieve a subset of the selected/requested diagnostic information to be included in the message.

In step 322, a diagnostic message can be generated. As an example, the diagnostic message can comprise the portion of the selected diagnostic information. However, any information can be included. In an aspect, the user settings 118 associated with an intended recipient of the diagnostic message are applied to the selected/requested diagnostic information. For example, a particular user setting 118 may include pre-defined information fields to be included in the messages that are sent to the user/recipient associated with the particular user setting 118. Accordingly, even though the selected/requested diagnostic information includes different information, the user setting 118 can take priority to retrieve a subset of the selected/requested diagnostic information to be included in the message.

In an aspect, computing device 104 and/or database 114 can comprise specialty templates for the messages. The specialty templates can be specifically created for each specialty in a particular profession, such as medicine/healthcare. For example, an orthopedic template can consist of basic patient information as well as x-rays. As a further example, a cardiology template can consist of basic patient information as well as an EKG or echocardiogram. As a further example, a gastroenterology template will consist of basic information as well as an abdominal x-ray or abdominal CT and perhaps lab information. The specialty templates can be created prior to implementation of the specific specialty message generation. Templates can be changed as needed. In an aspect, the requester of a message can select a pre-defined template for sending to an intended recipient.

In step 324, the message (e.g., diagnostic message) can be transmitted. As an example, the message can be transmitted to a recipient identified by the requester of the message. The message can be transmitted over any communication path or network such as the Internet and/or mobile telephone network.

In an aspect, the computing device 104 or other device can track a status of one or more transmitted messages. As an example, the following information can be tracked, time stamped, and or stored: date/time the message was generated/sent/received/forwarded/replied to; location (GPS) of requester/recipient; recipient device; receipt confirmation; read confirmation; no-read alert/feedback; and returned message alert. Other information and a feedback can be tracked/stored.

In an aspect, the scheduling application (e.g., Photon scheduling) can allow the client device 102 to set up appointments at a user's/consultant's office (e.g., office system 125). As an example, the client device 102 by accessing the scheduling data 130. As a further example, the user can reserve at least a portion of an available schedule for a particular set of patients (e.g., emergency room patients). In aspect, an emergency room provider, after requesting a diagnostic message, can use the scheduling application to send the diagnostic message directly to the office system 125 for direct uploading of diagnostic information into the user's office EMR software and provide the patient the appointment time, from the emergency room.

FIG. 3C illustrates an exemplary method for managing a diagnostic data based upon recipient availability. In step 332, a first message (e.g., diagnostic message) can be received by one or more users. As an example, the first message can comprise diagnostic information (e.g., injury description, images) to be reviewed by the recipient. As a further example, the first message can comprise a notification to retrieve diagnostic information from a remote location. In an aspect, the first message can be received by the client device 102. As an example, a confirmation message can be transmitted to a sender indicating whether the diagnostic message is received by a recipient device.

In another aspect, the user device 124 can be disposed in a location that is not equipped to communicate directly with the computing device 104. As an example, the client device 102 can operate as a proxy for the user device 104 when communicating with the computing device 104, the information system 108, and/or an office system 125. As a further example, the user device 124 may not be authenticated with one or more of the computing device 104, the information system 108, and/or an office system 125. As such, the user device 124 can communicate information to the client device 102 using a unique identifier (e.g., temporary or persistent) and the client device 102 can communication with one or more of the computing device 104, the information system 108, and/or an office system 125 on behalf of the user device 124. In an aspect, a user can locate contact information for a particular on-call physician. As an example, the user can transmit information to the on-call physician using standard communication networks (e.g., cellular, IP, media messaging, etc.). As a further example, the information transmitted can be tagged with an anonymous identifier that can later be associated with a particular medical record or patient file in a secure environment.

In yet another aspect, the first message of step 332 can be received by the client device 102 (e.g., a device associated on-call physician). Accordingly, a user of the client device can review the information comprised in the first message and can determine if the subject patient to whom the information relates should be transferred to a facility (e.g., emergency center, hospital, specialists office, etc.) for treatment and/or further analysis.

In step 334, a second message (e.g., diagnostic message, follow-up message) can be received by one or more users. As an example, the second message can comprise diagnostic information (e.g., injury description, images, supplemental information) to be reviewed by the recipient. As a further example, the second message can comprise a notification to retrieve diagnostic information from a remote location. In an aspect, the second message can be received by the client device 102. As an example, the second message can comprise supplemental information relating to the information comprised in the first message.

In step 336, one or more response options can be provided to the user. In an aspect, the response options can comprise one or more of a pre-defined diagnosis option, a forwarding the diagnostic message option, a communicating with sender option, and a generating a custom diagnosis option. Other options can be provided, such as an input option to allow the user to provide a particular response. As an example, at least one of the response options is customized based upon the diagnostic information.

In step 338, a selection of one of the plurality of response options can be received. In an aspect, the user can provide a particular information (e.g., consultation information, responses, feedback) to be included in a response message. As an example, the selection/provision of response information is received, for example, by the computing device 104. As a further example, the computing device 104 can process the response information to route the response to an appropriate recipient such as the client device 102.

In step 340, information, such as a response message (e.g., responsive diagnostic message), can be transmitted based upon the selection of the one of the plurality of response options. In an aspect, the message can be transmitted to the requester of the diagnostic information. As an example, the message can be transmitted or forwarded to a user for further review and consultation. As a further example, the message can be transmitted to the office system 125, wherein, for example, the information in the message can be used to update records stored in the office system 125.

In an aspect, in response to receiving the first message of step 332, the recipient can recommend that the subject patient be admitted in an emergency center. As an example, the subject patient can be admitted or checked-in to the emergency center. Detailed patient information can be collected and stored. Tests and procedures can be performed, such as X-rays and MRI's and the collected information can be electronically stored. In another aspect, a medical record or patient record can be created. As an example, the information comprised in the first message can be linked or associated with the newly created medical record, for example, by associated the anonymous identifier with the medical record. As a further example, information from one or more of the first message and the second message can be merged into a single record, such as a secure PDF. In an aspect, records can be stored on a secure HIPAA compliant server. As an example, a location such as a filename or URL can be used to identify and distinguish between records. Accordingly, the record can be subsequently accessed using the identifier.

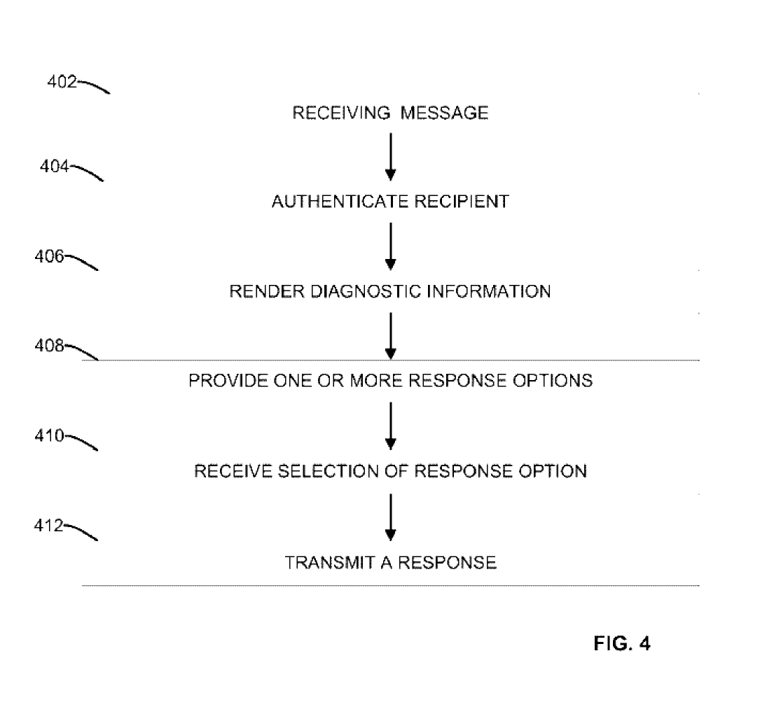

FIG. 4 illustrates an exemplary method for managing received diagnostic message. In an aspect, in step 402, a message (e.g., diagnostic message) can be received by one or more users. As an example, the message can comprise diagnostic information to be reviewed by the recipient. As a further example, the message can comprise a notification to retrieve diagnostic information from a remote location. In an aspect, the message can be received by the user device 124. As an example, a confirmation message can be transmitted to a sender indicating whether the diagnostic message is received by a recipient device.

In step 404, the diagnostic information can be rendered to the user. In an aspect, a recipient can be authenticated prior to rendering the diagnostic information. As an example, the diagnostic information can comprise audio, text, images, and video. As a further example, the recipient/user can interact with the user device 124 to review the diagnostic information. In an aspect, the diagnostic information can be classified and/or organized by pre-defined categories, such as objective, test results, images, historical information, or the like. As an example, a confirmation message can be transmitted to a sender indicating whether the diagnostic message has been reviewed by a user.

In step 406, one or more response options can be provided to the user. In an aspect, the response options can comprise one or more of a pre-defined diagnosis option, a forwarding the diagnostic message option, a communicating with sender option, and a generating a custom diagnosis option. Other options can be provided, such as an input option to allow the user to provide a particular response. As an example, at least one of the response options is customized based upon the diagnostic information.

In step 408, a selection of one of the plurality of response options can be received. In an aspect, the user can provide a particular information (e.g., consultation information, responses, feedback) to be included in a response message. As an example, the selection/provision of response information is received, for example, by the computing device 104. As a further example, the computing device 104 can process the response information to route the response to an appropriate recipient such as the client device 102.

In step 410, information such as a message can be transmitted based upon the selection of the one of the plurality of response options. In an aspect, the message can be transmitted to the requester of the diagnostic information. As an example, the message can be transmitted or forwarded to a user for further review and consultation. As a further example, the message can be transmitted to the office system 125, wherein, for example, the information in the message can be used to update records stored in the office system 125.

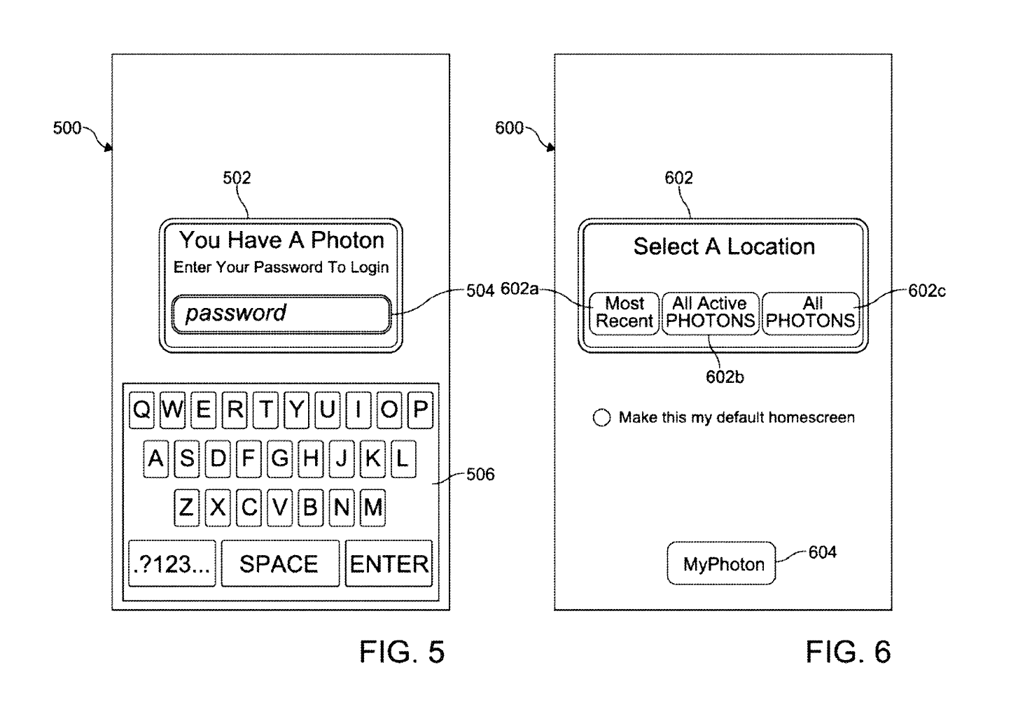

In an aspect, FIG. 5 illustrates a user interface 500 comprising a message notification 502. As an example, a user must be authenticated prior to viewing the message. As a further example, the user interface 500 can comprise a password input 504 for receiving a password to authenticate the user/recipient. In an aspect, the user interface 500 can comprise a keyboard or keypad to receive an input from the user (e.g., to ensure HIPAA compliance).

In an aspect, FIG. 6 illustrates a user interface 600 comprising a menu 602. As an example, the menus 602 can comprise options for navigating and organizing one or more diagnostic messages. In an aspect, a plurality of diagnostic messages can be organized by most recent 602a, all active messages 602b (e.g., message that require action), and/or all photons 602c. As an example, the interface 600 can comprise a homepage button 604, wherein a user can adjust settings.

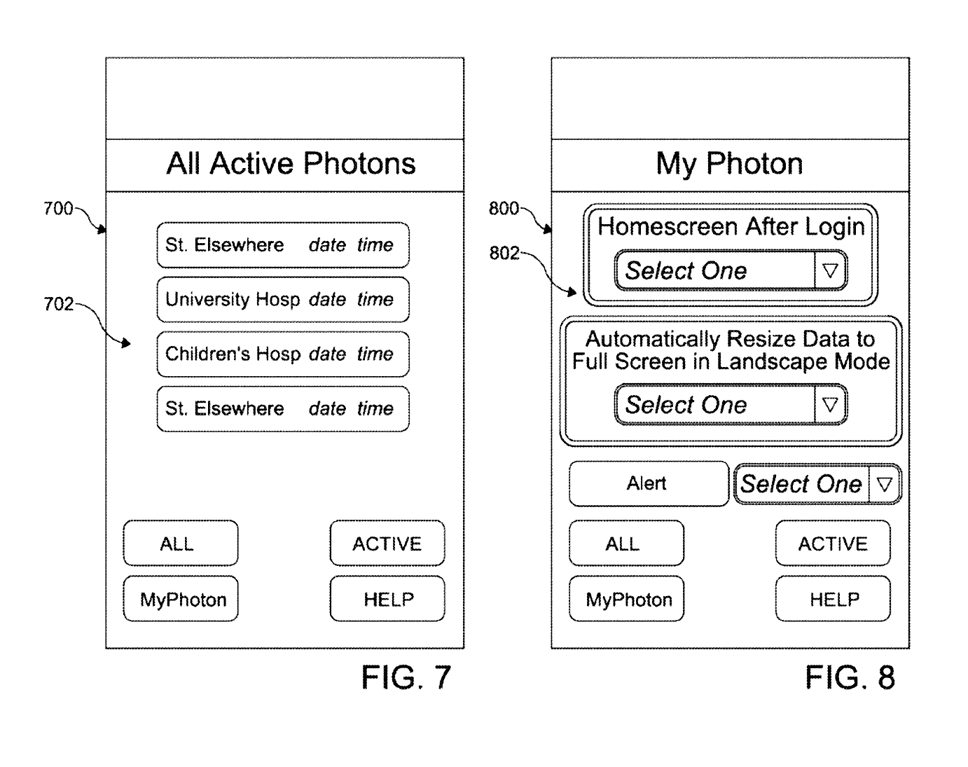

In an aspect, FIG. 7 illustrates a user interface 700 comprising a plurality of active messages icons 702 representing messages that require action. As an example, the messages can comprise sender information, a date, and a time. Other information can be presented by the message icons.

In an aspect, FIG. 8 illustrates a user interface 800 comprising a plurality of setting options 802. As an example, the settings options can be presented as part of a “My Photon” page or homepage.

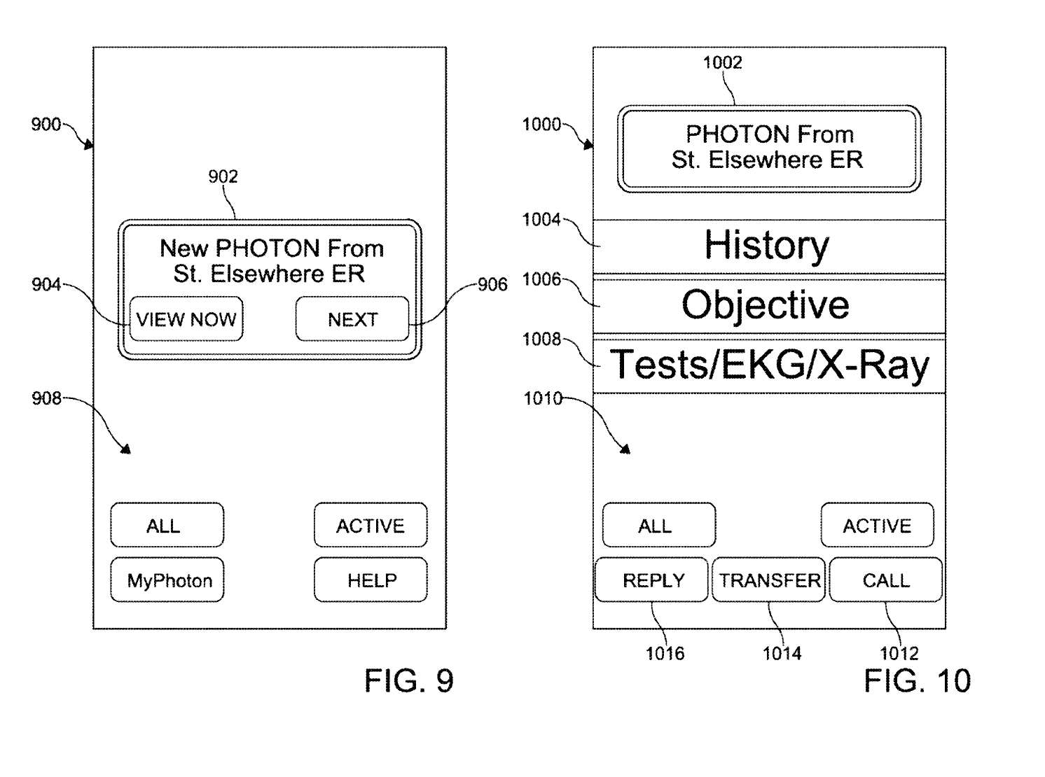

In an aspect, FIG. 9 illustrates a user interface 900 comprising an active message notification 902. As an example, the notification 902 can comprise options, such as view now 904 and/or a scrolling option (e.g., next 906) to facilitate the navigation through a plurality of notifications. As a further example, the user interface 900 can also comprise universal options 908 to facilitate navigation between various user interface screens.

In an aspect, FIG. 10 illustrates a user interface 1000 comprising a message header 1002 identifying the particular message. The user interface 1000 can comprise diagnostic information, wherein the diagnostic information can be organized or categorized. As an example, the categories can comprise history 1004, objective 1006, test/EKG/X-rays 1008 and/or other classifications. As a further example, a plurality of response options 1010 can be provided to the user. In an aspect, the response options 1010 can comprise call 1012, transfer 1014, and/or replay 1016. Other options can be provided.

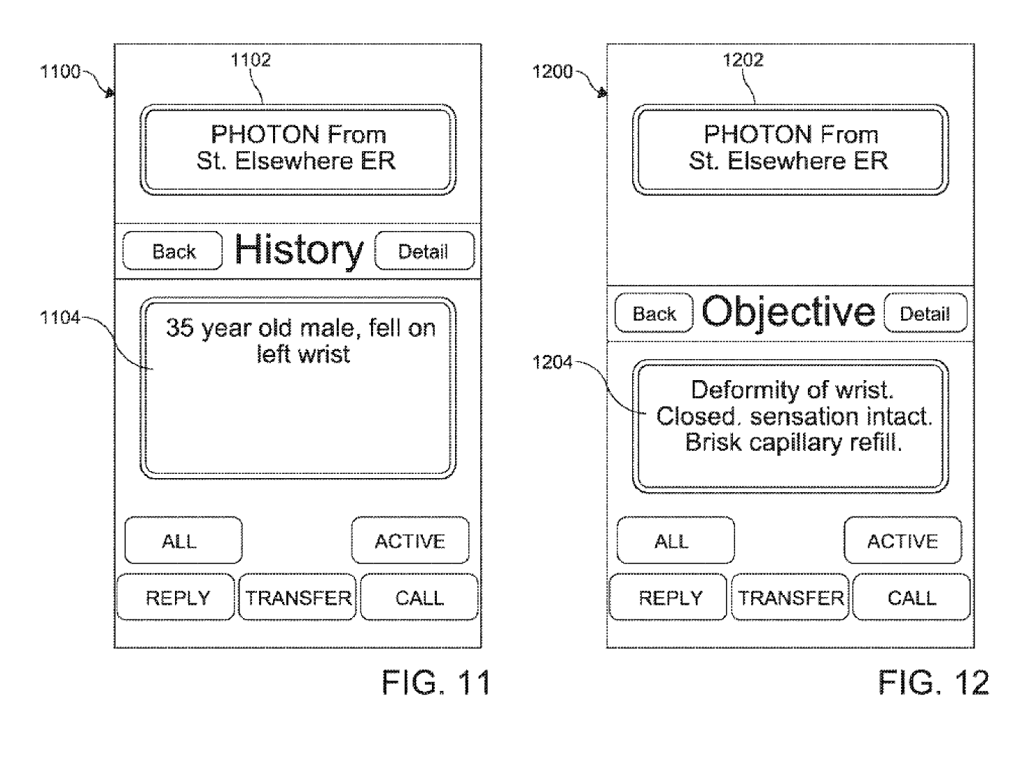

In an aspect, FIG. 11 illustrates a user interface 1100 comprising a message header 1102 identifying the particular message. In an aspect, the user interface 1100 can comprise historical information 1104, such as a medical history, a background to a particular incident, and/or past diagnostic information. Other information can be provided.

In an aspect, FIG. 12 illustrates a user interface 1200 comprising a message header 1202 identifying a particular message. In an aspect, the user interface 1200 can comprise objective information 1204, such as diagnostic information, medical information, and/or a background to a particular incident. Other information can be provided.

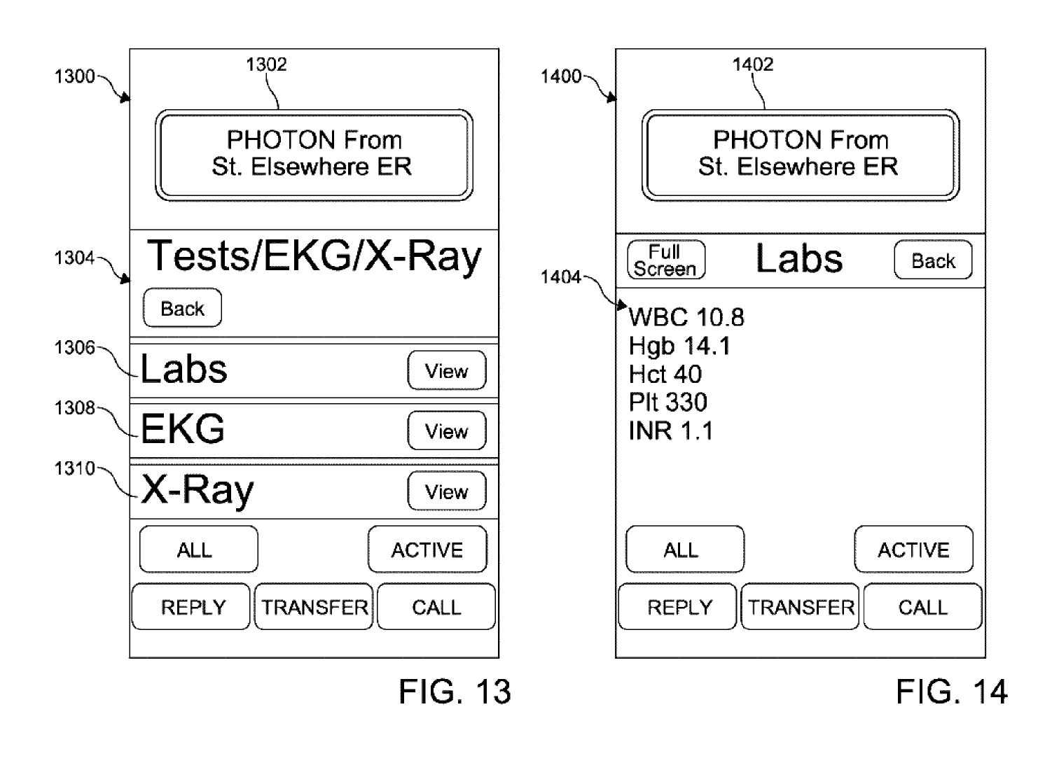

In an aspect, FIG. 13 illustrates a user interface 1300 comprising a message header 1302 identifying a particular message. In an aspect, the user interface 1300 can comprise diagnostic information 1304, such as lab results 1306, EKG 1308, and/or X-ray 1310. Other information can be provided.

In an aspect, FIG. 14 illustrates a user interface 1400 comprising a message header 1402 identifying a particular message. In an aspect, the user interface 1400 can comprise detailed lab information 1404, such as lab results or other test information. Other information can be provided.

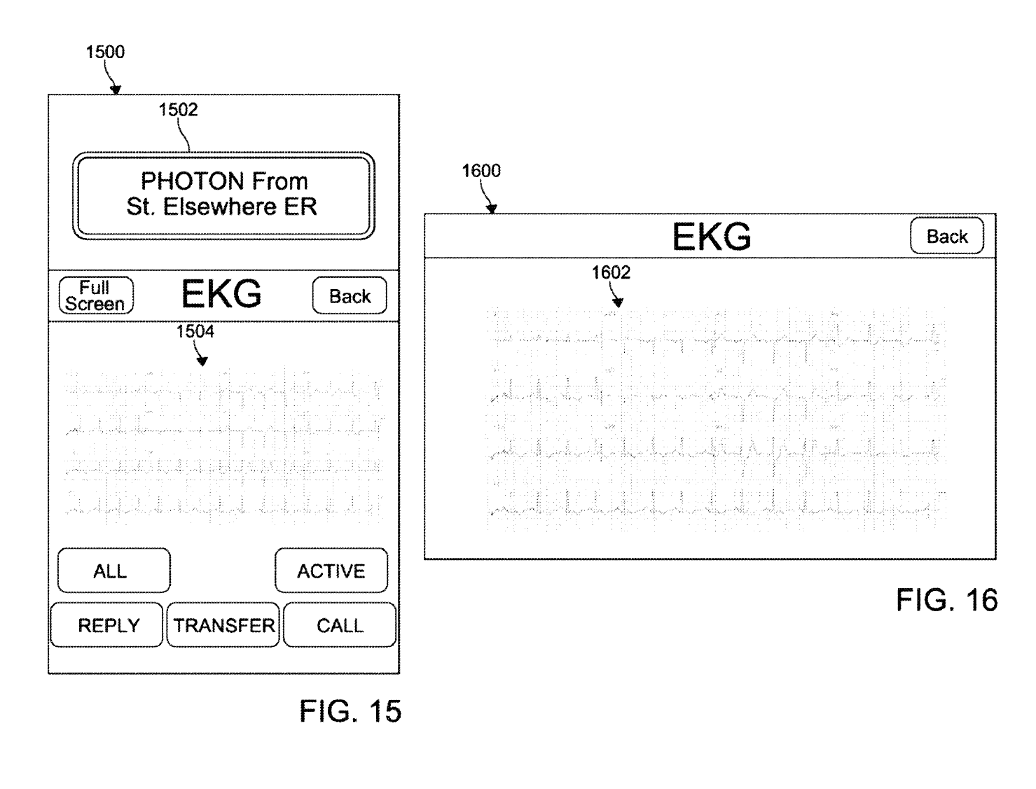

In an aspect, FIG. 15 illustrates a user interface 1500 comprising a message header 1502 identifying a particular message. In an aspect, the user interface 1500 can comprise detailed monitoring information 1504, such as EKG or other test information. Other information can be provided. In an aspect, FIG. 16 illustrates a user interface 1600 can render an enlarged image 1602 representing a test result or medical information.

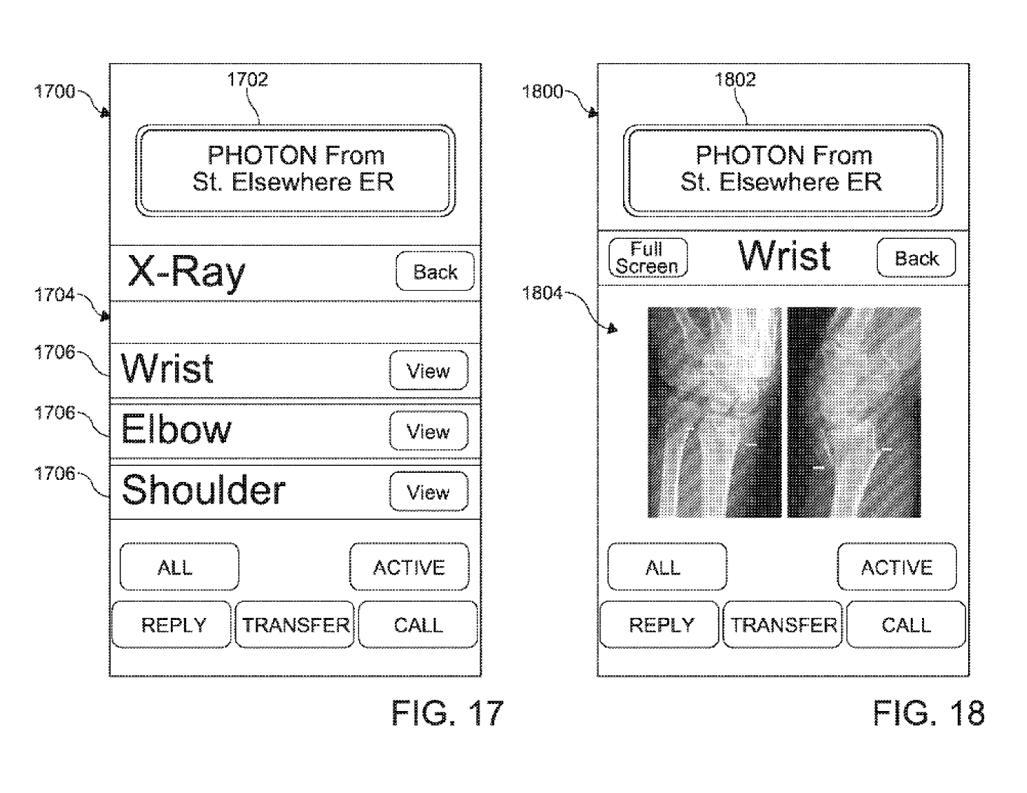

In an aspect, FIG. 17 illustrates a user interface 1700 comprising a message header 1702 identifying a particular message. In an aspect, the user interface 1700 can comprise detailed imaging information 1704, such as X-rays or other test information. Other information can be provided. In an aspect, a plurality of images 1706 can be classified and rendered for a user to view.

In an aspect, FIG. 18 illustrates a user interface 1800 comprising a message header 1802 identifying a particular message. In an aspect, the user interface 1800 can comprise a rendered image 1804, such as an X-ray or other test information. Other information can be provided.

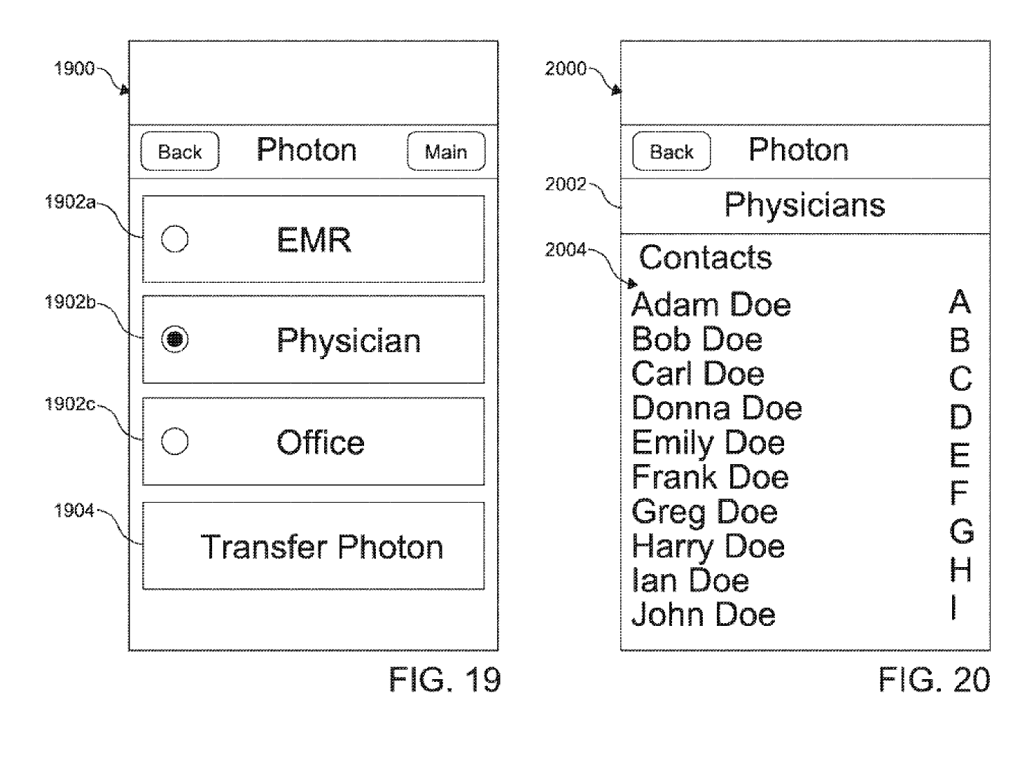

In an aspect, FIG. 19 illustrates a user interface 1900. In an aspect, a user can select to transfer or forward the diagnostic message and/or diagnostic information to another user or recipient. As an example, the user interface 1900 can provide forwarding options and/or selectable destinations such as an EMR 1902a, a physician 1902b, an office 1902c (e.g., the office system 125), or other destination. As a further example, the user interface 1900 can comprise a confirmation button 1904 for initiating the transfer once a intended recipient has been provided.

In an aspect, FIG. 20 illustrates a user interface 2000 comprising a contact list, including a contact classification header 2002 and an organized list 2004 of potential recipients of a forwarded message and/or information.

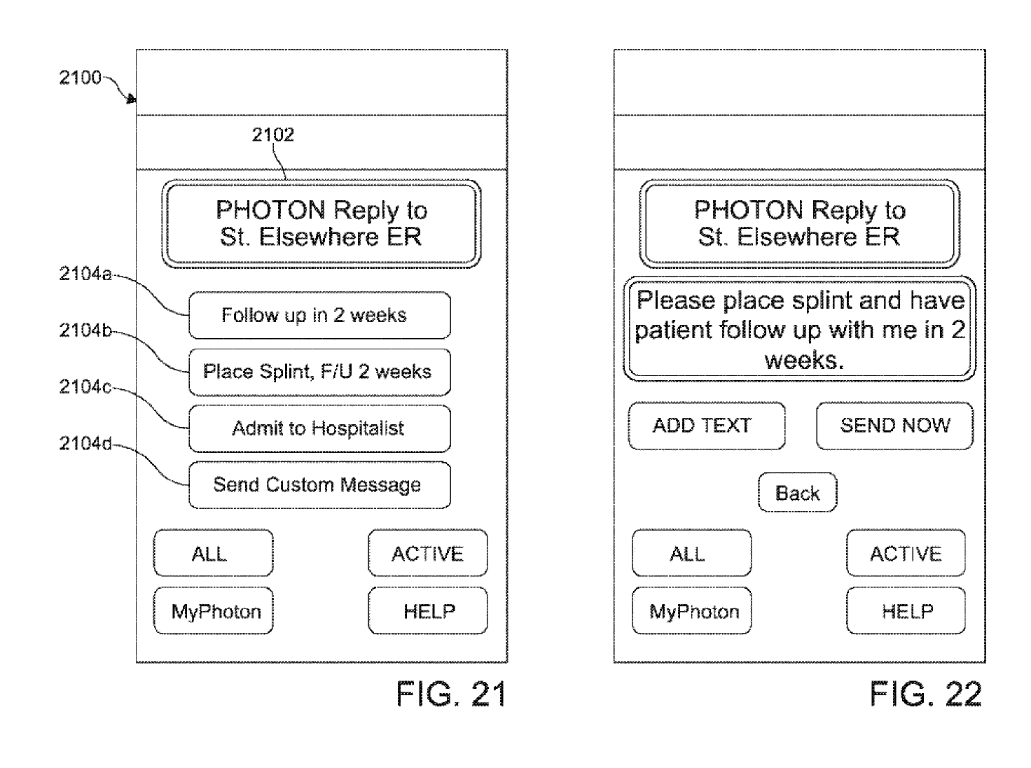

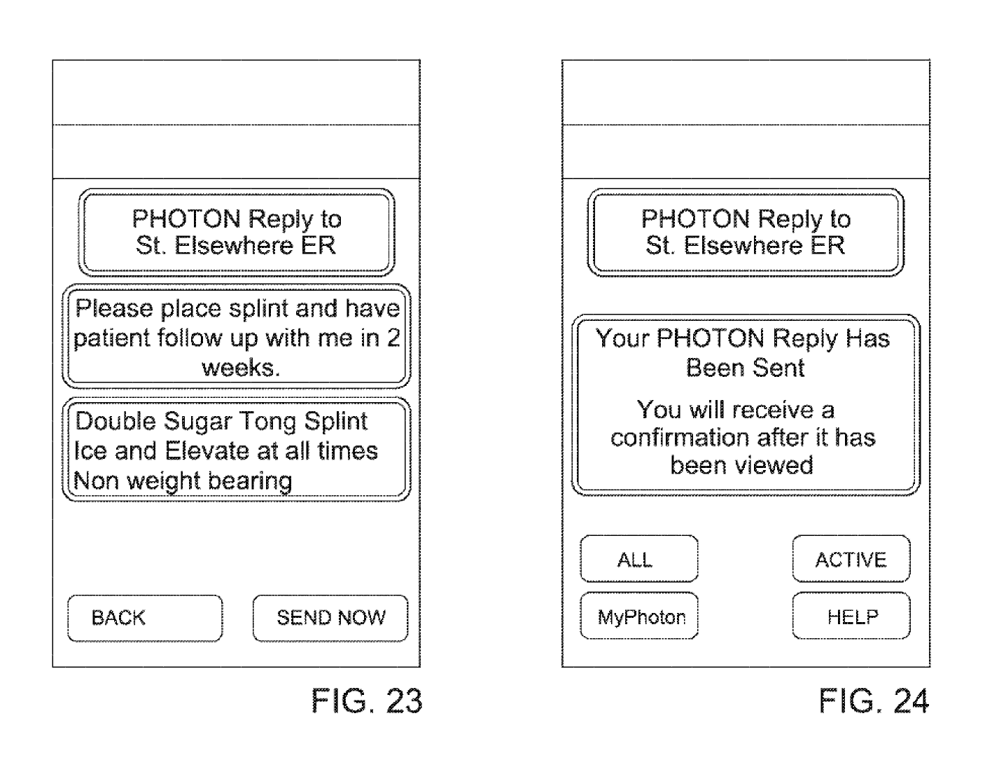

In an aspect, FIG. 21 illustrates a user interface 2100 comprising a message header 2102 identifying a particular message. In an aspect, a user can select to provide a replay to the diagnostic message. As an example, the user interface 2100 can comprise pre-defined response options 2104a, 2104b, 2104c, 2104d. In an aspect, the response options 2104a, 2104b, 2104c, 2104d can be defined based upon the diagnostic information included in the message. As an example, the response options 2104a, 2104b, 2104c, 2104d can comprise a customizable response option 2104d to allow a user to create a customized response message, as shown in further detail in FIGS. 22-23. In an aspect, once a response message has been generated (FIG. 23), the response message can be transmitted to a recipient. As an example, a confirmation can be provided to the user, as shown in FIG. 24, thereby completing an exemplary cycle of consultation.

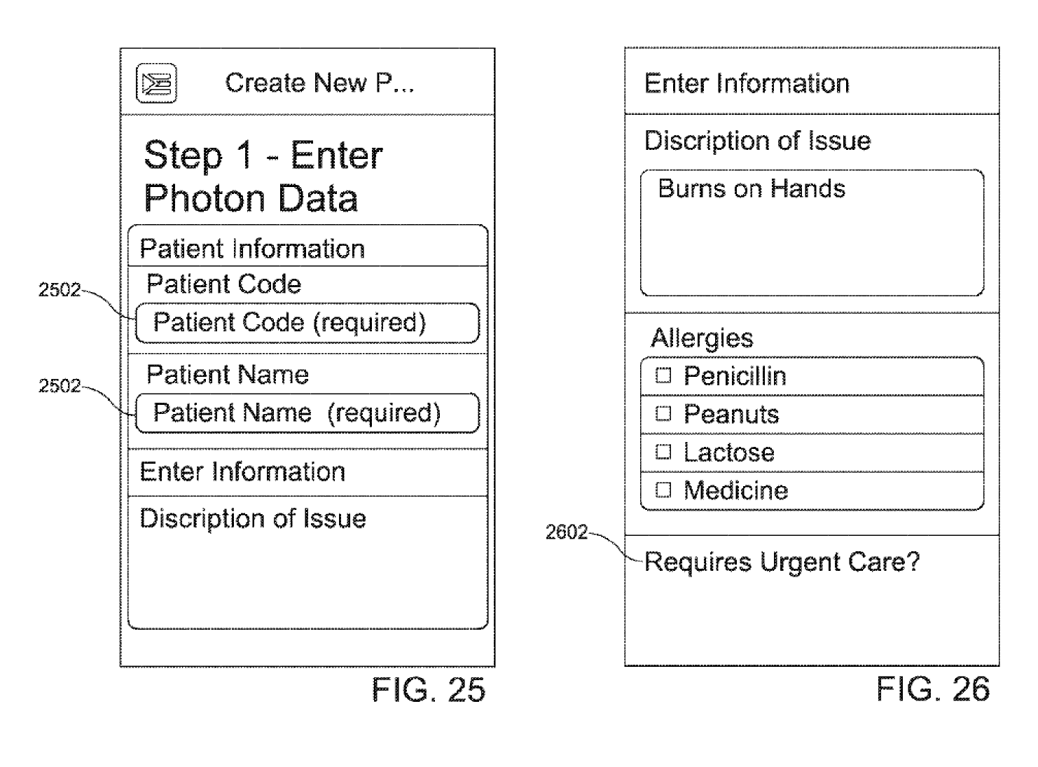

FIG. 25 illustrates a messaging screen (e.g., a Photon lite screen). In an aspect, the messaging screen can facilitate secure and unsecure communication of information to one or more devices such as client devices 102 and/or user devices 124, for example. In another aspect, the messaging screen can be rendered via user device 124 that is disposed in a location that is not equipped to communicate directly with the computing device 104. As an example, the client device 102 can operate as a proxy for the user device 104 when communicating with the computing device 104, the information system 108, and/or an office system 125. As a further example, the user device 124 may not be authenticated with one or more of the computing device 104, the information system 108, and/or an office system 125. As such, the user device 124 can communicate information to the client device 102 using a unique identifier (e.g., temporary or persistent) and the client device 102 can communication with one or more of the computing device 104, the information system 108, and/or an office system 125 on behalf of the user device 124. In an aspect, a user can locate contact information for a particular on-call physician. As an example, the user can transmit information to the on-call physician using standard communication networks (e.g., cellular, IP, media messaging, etc.). As a further example, the information transmitted can be tagged with an identifier (e.g., anonymous) that can later be associated with a particular medical record or patient file in a secure environment.

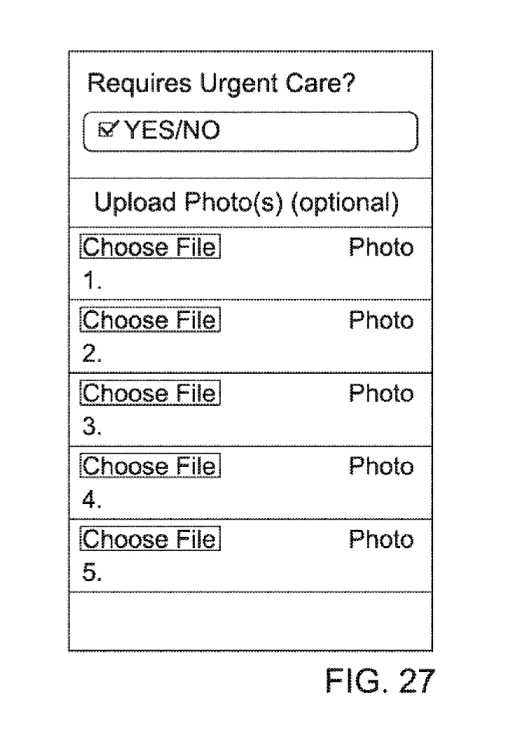

In an aspect, a patient code 2502 can be provided (e.g., manually or automatically) as an identifier for tracking information associated with the patient code 2502. As an example, a patient name 2504 can be provided (e.g., inputted), as opposed to pulling patient information from the hospital EMR. As a further example, information such as a description of the patient or medical issue/condition can be provided. In another aspect, an urgency option 2602 can be provided, as shown in FIG. 26. As an example, the urgency option 2602 can be rendered to a user of the messaging screen, whereby a selection of the urgency option indicates a need for immediate care of the subject of the message. As a further example, description information and/or the urgency option 2602 can be associated with the patient code 2502 for associating the images with a particular patient. In a further aspect, one or more images can be provided (e.g., loaded, attached to the message, etc.), as shown in FIG. 27. As an example, a camera can be used to capture an image. The image can comprise video, audio, still images, or the like. Captured images can be encrypted and not saved to the device in a readable manner (or stored in a secure hidden directory). Once the capture images are transmitted, the images can be removed from the capturing device. As a further example, one or more captured images can be associated with the patient code 2502 for associating the images with a particular patient.

In an aspect, the systems and methods described herein provide a simple “click-by-click” process for both the emergency room provider and the consultant. With regards to the emergency room physician, once the scheduling packages open, a first click can comprise opening an orthopedic call schedule. A second click can comprise sending a Photon to the orthopedist that is on call, thereby resulting in a two-click process. With regards to the consultant physician, once the consultant has received notification that the Photon is now on a smart device, the consultant can log into an interface application (e.g., the Photon application), constituting a first click. If there is only one Photon available, it will automatically be populated. The consultant can be able to review all the relevant information instantly. The consultant can then click on an action button or transmit button to send the appropriate response, thereby constituting a second click. However, automation of certain events, such as push notifications, populating an interface, displaying options, and the like can facilitate a single click operation.

In an aspect, the systems and methods disclosed herein can be integrated with one or more scheduling products to facilitate a compliant scheduling program comprising the ability to send compliant images and messages bidirectionally, and save such messages into an EMR. As an example, users can use Photons to transmit a “facesheet” for billing, wherein the can comprise a patient's demographics, insurance information, contact information.

Photons can be sent to virtually anyone, from a health care provider, to industry (e.g., parsing out non-HIPAA compliant information). As an example, in-theatre military healthcare applications can leverage the benefits of the systems and methods disclosed herein.

### Patient-Centric Management

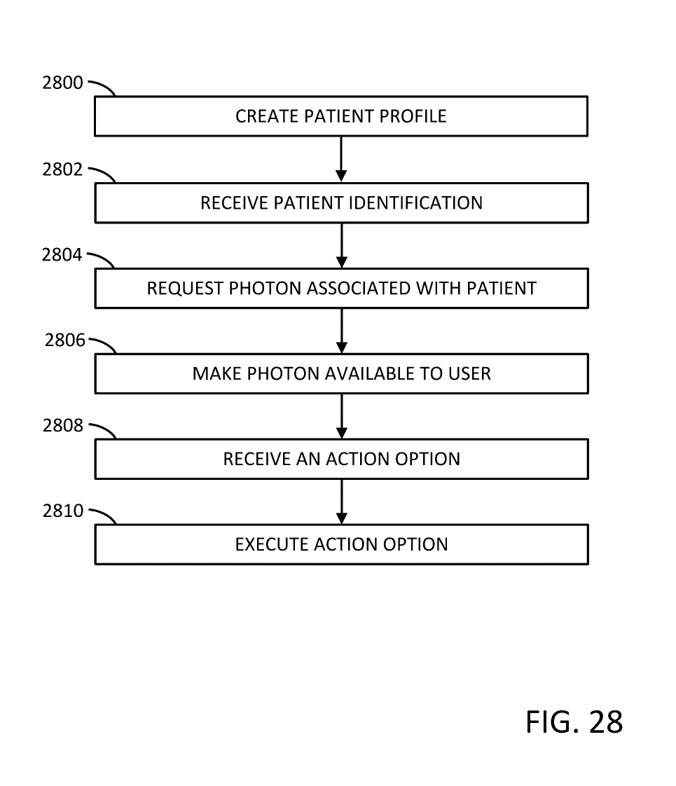

In an aspect, FIG. 28 illustrates an exemplary method for executing an action related to the care of a patient. Although the term patient is used in reference to the method shown in FIG. 28, it should be understood that the patient can be any person such as a recipient of service (e.g., a client, customer, participant, or other classification of person receiving service).

In step 2800, a patient profile can be created. In an aspect, a user can be authenticated using login credentials to access administrative options associated with the computing device 104 or a system associated with the computing device 104. Once authenticated, the user can create a patient profile by providing patient information such as contact information, medical information, insurance information, preferences, and other information relating to patient and/or treatment, for example. Such information can be populated from a patient record (e.g., patient record 119) once the user identifies a patient identifier. Such information can be manually entered by the user or loaded from a data source. The patient profile can be linked to one or more users (e.g., physicians, nurses, service providers, entities, etc.) based upon preset rules, identified relationships, or associations identified by the patient information. As an example, each physician that has a relationship with the identified patient in the patient profile can be linked to the patient profile. Such linkage can be manually established or automated. Through such linkage, any information relating to the patient profile can be transmitted (e.g., broadcast) to each of the users linked to the profile. Any number of users can be linked to any number of patient profiles. Any updates to the patient profile can be transmitted to the linked users. As another example, the patient profiles can be stored, for example, in the database 114. Other information can be stored in the database 114 and/or associated with a particular patient profile. For example, a patient record 119 can be linked to the patient profile and the profile can be populated with information from the linked patient record 119.

In operation, the patient profiles facilitate the patient-centric management of a patients information and service (e.g., healthcare). For example, using the patient profile, a patient can be admitted, transferred, discharged, scheduled, and/or diagnosed by one or more users linked to the patient profile. In an aspect, checklists and clearances can be managed using the patient profiles, whereby one or more users can access the profile to determine if certain tasks have been completed relating to the patient. As an example, a surgical clearance may require lab results, X-ray, consent forms, and anesthesiologist review and such clearance tasks can be centrally managed via the patient profile. In this way, any linked user to the patient profile can understand which tasks are complete and whether the patient is cleared for surgery.

In step 2802, identification of a patient can be received. In an aspect, a user can identify a patient for whom the user wishes to perform an action and such identification can be received by a computing device such as computing device 104. As an example, the user can select a patient profile (e.g., via a displayed menu). As an example, the user can be presented with a user interface (e.g., via user device 124 or client device 102) through which the user can input the patient's name or identification number or select the patient from a grouping of patients (e.g., populated graphically from a server). The grouping of patients can be based on a set of one or more patients associated with the user or a set of one or more patients associated with the user's facility, office, practice, service specialty, service region, etc., for example. The group of one or more patients can be generated based upon the linkages between the particular user and one or more patient profiles. As an example, the user can select the patient via user device 124 or client device 102. However, other devices and systems can be used to select the patient.

In an aspect, in step 2804, a Photon associated with the identified patient can be requested from a computing device 104. As an example, the request can be transmitted over any communication path or network such as the Internet and/or mobile telephone network. The computing device 102 can then retrieve the Photon or information relating to the request from any storage medium. As an example, at least a portion of the requested Photon information is retrieved from the database 114. The computing device 102 can also retrieve requested Photon information from an information system 108, for example. Photon information from an information system can include, as examples, imaging data 110 or scheduling data 112 from that information system. As a further example, the computing device can receive requested Photon information from an office system 125. Photon information from an office system can include, as examples, patient data 128 or scheduling data 130 from that office system. In an aspect, the user settings 118 associated with the user are applied to the requested Photon information. For example, a particular user setting may prohibit the user's access to some patient information within the Photon. Accordingly, the rules engine 122 can filter out the prohibited patient information from the Photon that is transmitted.

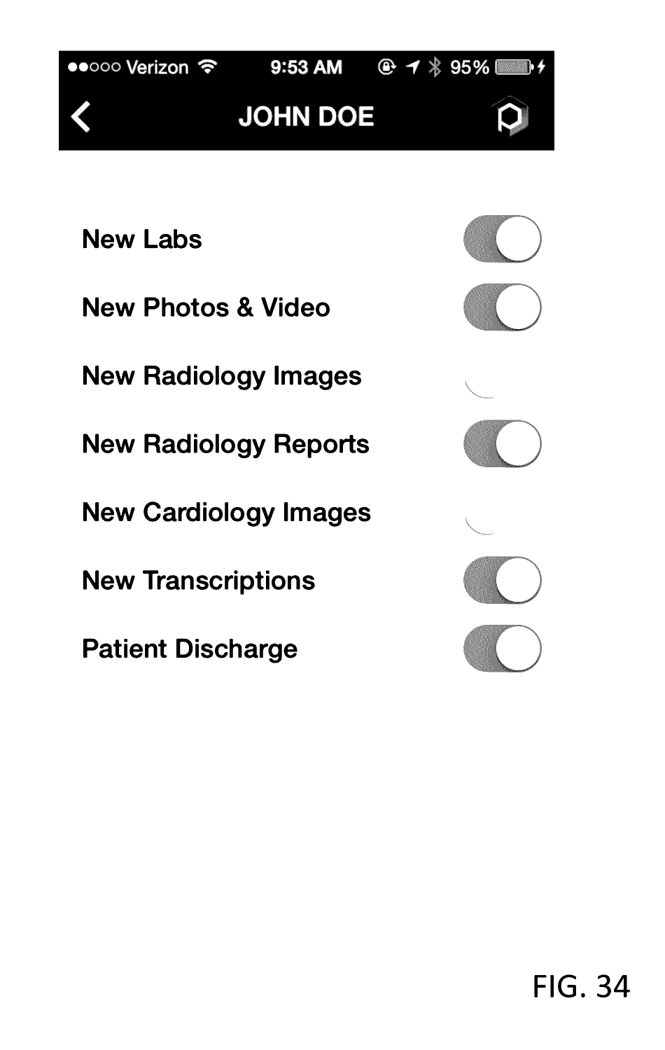

In step 2806, the requested Photon can be made available to one or more users. In an aspect, a notice can be transmitted to the users linked to patient profile associated with the requested Photon. The notice can indicate to the recipient that information is available to the recipient. As an example, the recipient user can then access the Photon, for example via the user device 124 or client device 102. As an example, the Photon notification and/or displayed information can be transmitted over any communication path or network such as the Internet and/or mobile telephone network. As a further example, the Photon can be accessed via an HTTP request, whereby information is securely presented to the requester without storing such information to the local requesting device. As an example, notification settings can be toggled on/off on a per patient basis, per entity basis, per user basis, and the like, as shown in FIG. 34.

In step 2808, an action option can be received. In an aspect, the user can be presented with an interface containing a plurality of action options pertaining to the identified patient. As examples, the interface could include one or more of buttons, drop-down menus, and expandable lists. One example of an action option is an option to create a new record for the patient. A record can include any information about the patient, including, but not limited to, a record of an office visit, a record of a hospital visit, physician notes, lab results, or radiology imagery such as an X-ray, CT scan, or MRI. Another example of an action option is an option to view the patient's information. An example of patient information can be the patient's biographical information. Biographical information can include, but is not limited to, name, weight, height, ethnicity, date of birth, address, phone number, current medical conditions, past medical conditions, current medications, past medications, and insurance information. Another example of patient information can include information on the patient's healthcare providers. Provider information can include, but is not limited to, information on the patient's current primary care physician, past primary care physicians, current specialists, past specialists, current nurse, and past nurses. Another example of patient information can be the patient's records. A record can include any information about the patient, including, but not limited to, a record of an office visit, a record of a hospital visit, physician notes, lab results, or radiology imagery such as an X-ray, CT scan, or MRI.

Another example of an action option is an option to transfer care of the patient. An option to transfer the patient can include transferring the care of the patient to another healthcare provider, such as another doctor or nurse. This option can also include transferring the patient to another healthcare facility, such as another hospital or long-term care home, which will then assume care of the patient. This option can further include transferring the patient within a healthcare facility to another department or unit within the healthcare facility. For example, a patient can be transferred from an intensive care unit to a standard care unit within the same hospital. Another example of an action option is an option to admit a patient to a healthcare facility. Another example of an action option is an option to discharge a patient from a healthcare facility.

Another set of examples of action options include options pertaining to scheduling. For example, an option can be to view the patient's healthcare schedule. The healthcare schedule can include, but is not limited to, any upcoming procedure, surgery, physician office visit, physical therapy, occupational therapy, lab test, or diagnostic. The option to view the healthcare schedule can also include viewing the past schedule of the patient. As another example, an option can be to schedule an appointment for the patient. The appointment can be for, as examples, a procedure, surgery, physician office visit, physical therapy, occupational therapy, lab test, or diagnostic. In an aspect, when an appointment for a procedure or surgery is scheduled, the interface presents the user with a checklist of requirements that must be completed before the procedure or surgery can be performed. Examples of requirements that a checklist can contain include whether all necessary lab work has been completed; whether all necessary diagnostics (e.g., X-rays, biopsies) have been completed; whether the patient has signed the necessary consent forms; and whether an anesthesiologist is scheduled and has given his or her approval for the procedure or surgery. If one or more checklist items are not complete, the user can have an option to send a notification to the party responsible for that task. For example, if a surgery cannot be performed until an X-ray is completed; the user can send a notification to the radiology unit instructing them to perform the X-ray.

In another aspect, when the user schedules an appointment, the system communicates a request to the relevant information system 108 or office system 125 to return scheduling data 112, 130. When the scheduling data is returned, the system determines if there is an available appointment. In an aspect, the system schedules the appointment and communicates the appointment information back to the relevant information system 108 or office system 125, which then updates the respective scheduling data 112, 130. The communication between the user device 124 or client device 102 and the information system 108 or office system 125 can be direct, via a network 100, and/or via the computing device 104. In the context of a primary care physician office visit, for example, this action serves to check to see when the office has free appointment slots. The user may then select an open slot and the selection is communicated back to the office, thus securing the patient his or her office visit appointment. In the context of a hospital surgery, for example, this action can function in a similar manner as to that for a primary care physician office visit, but it can also check that the necessary support staff, such as nurses and an anesthesiologist, is available.

Another example of an action option is an option to send a message. This option can further include options to send a message to one or more providers already associated with the patient. For example, the user can choose to send a message to one provider, a subset of providers, or all providers associated with the patient. If the user wishes to send a message to a provider not already associated with the patient, the interface can include an option to manually enter a provider or select the provider from a list.

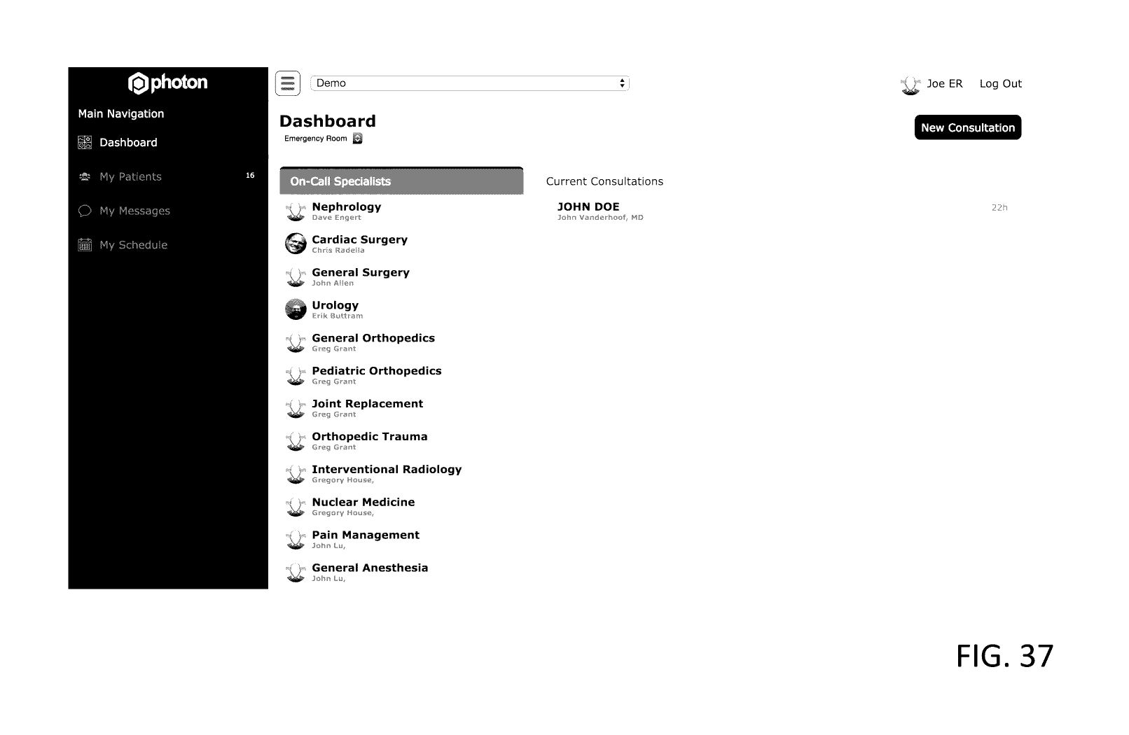

In step 2810, the selected action option can be executed (e.g., processed). The execution of the action option can include execution by a user device 124, client device 102, computing device 104, information system 108, office system 125, or a combination thereof. As an example, execution of an action option to view a patient's information may only involve execution on the local user device 124 or client device 102 since the device has already received the patient information in the Photon and it is merely a matter of displaying it to the user. As another example, execution of an action option to schedule an appointment for the patient at a physician's office may involve execution by both the local user device 124 or client device 102 and a remote office system 125. As an example, an emergency room dashboard is illustrated in FIG. 37 showing an integrated scheduling program and a ‘3 click process’ to generate a consult.

As an illustrative example, a computer-implemented method for providing patient-centric management of a patient service may comprise: generating, by one or more processors, a patient profile comprising a patient identifier and information relating to the patient identifier; linking one or more service providers of the plurality of service providers to the generated patient profile, wherein the one or more linked service providers are capable of accessing the patient profile over a secure network connection; receiving, by the one or more processors, an update relating to the patient profile; automatically transmitting, by the one or more processors, a notice to the one or more linked service providers, wherein the notice indicates that an update has been received relating to the patient profile; receiving, by the one or more processors, a request to access the patient profile from the one or more linked service providers; and granting access to the patient profile including the update, via a secure network connection.

The patient profile may comprises medical information relating to a particular patient. The medical information may be compiled internally to a closed system (e.g., EMR service or hospital) or may be compiled from at least two discrete healthcare systems.

Generating the patient profile may comprise receiving medical information and sorting the medical information based upon medical relevance. Medical relevance may be determined based on a set of predefined rules that may be specific to various conditions, identifiers, warnings, keywords, threshold conditions, or a combination of the same reflecting a predefined relevance signature for a particular treatment, or relevant specialist. Generating the patient profile may comprise receiving medical information and including only a subset of the received medical information in the patient profile, wherein the subset is based on a medical relevance.

The update to the patient profile may comprise receiving updated medical information such as additional patient information, tests, scans, diagnosis, or patient changes, or may comprise receiving a message relating to the patient profile. The message may be received from one of the linked service providers or an unlinked user provided with a limited credential (e.g., time to live credential with time expiration).

Granting access to the patient profile may comprise allowing a user to transmit a message to or receive a message from other users, wherein the message relates to the patient profile. The granting access to the patient profile may comprise transmitting medically relevant information to a user. The granting access may comprise providing credentials to an unlinked service provider, for example, on limited basis. In certain aspects, the communications between the linked service providers (or others) may be stored as part of the patient profile.

### Photon View

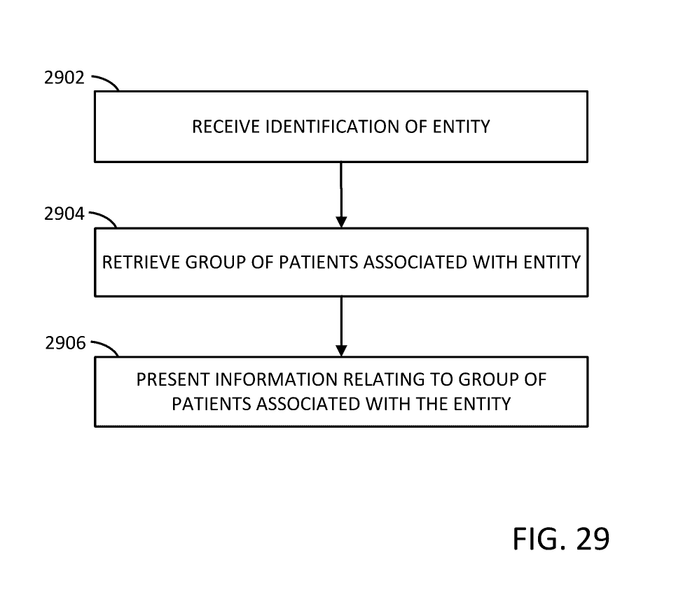

In an aspect, FIG. 29 illustrates an exemplary method for providing a grouping of patients associated with an entity or particular user. Although the term patient is used in reference to the method shown in FIG. 29, it should be understood that the patient can be any person such as a recipient of service (e.g., a client, customer, participant, or other classification of person receiving service).

In step 2902, an identification of an entity can be received. In an aspect, a user wishing to view all the patients associated with a particular entity can identify that entity to the computing device 104. As examples, the user can identify the entity by name, number, or code. As additional examples, the user can identify the entity from a list or drop-down menu of entities presented via device such as user device 124 or client device 102 and the information system 108 or office system 125. However, other devices and systems can be used to select the entity. An entity can be an individual healthcare provider, such as a physician, nurse, or therapist; an organization, such as a health care organization (HMO) or accountable care organization (ACO); a facility, such as a hospital or physician office; or any other association of providers.

In step 2904, a group of patients associated with the entity can be retrieved (e.g., from computing device 104). As an example, a request can be transmitted over any communication path or network such as the Internet and/or mobile telephone network to retrieve information relating to the group of patients from a remote system. As another example, the computing device 104 can retrieve information relating to the group of patients from any storage medium based upon, for example, a query of all patients associated with the entity. As an example, at least a portion of the requested information is retrieved from the database 114.

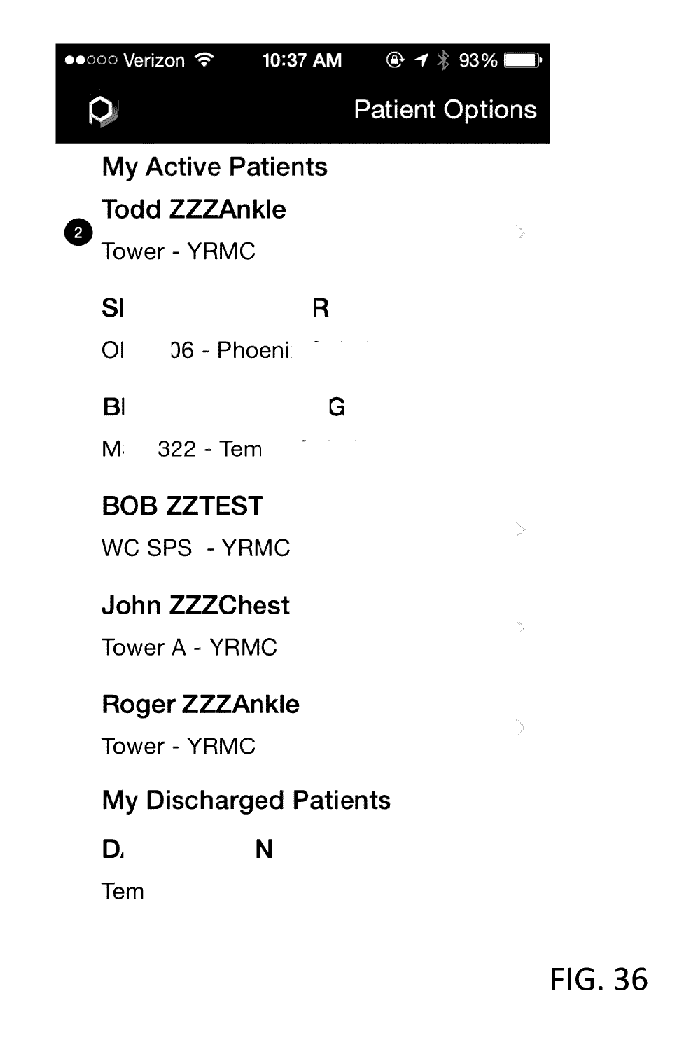

In an aspect, in step 2906, the information relating to the group of patients associated with the entity can be presented (e.g., made available, transmitted, displayed, etc.) to the user. As an example, the grouping of patients can be displayed as a text list, a graphical representation, or combination thereof. In an aspect, if the user wishes to access a Photon or perform an action with respect to one or more patients, the user can do so by selecting the one or more patients in the displayed list. As an example, a listing of patients is shown in FIG. 36.

### Courtesy Credentials

In an aspect, secure credentials for access information and/or a communication session relating to a patient can be proxied to one or more users or entities as a courtesy credential. Such courtesy credential can allow a non-credentialed entity to have access to certain information via the use of a credentialed party. Courtesy credentials can provide full access or can be limited based on various rules and settings that can be set by an administrator or the credentialed party.

### Role-Based Modes

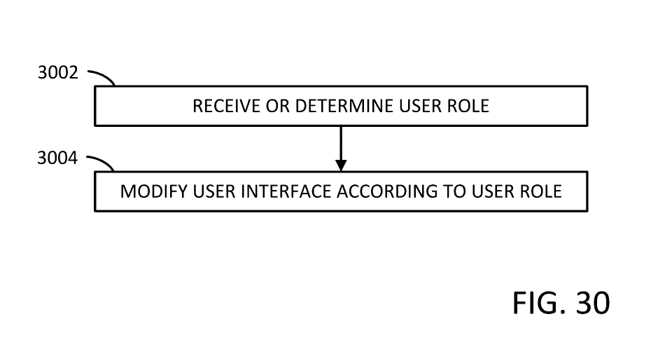

FIG. 30 illustrates an exemplary method for modifying a user interface and functions related thereto, according to a user's role. Although the term patient is used in reference to the method shown in FIG. 30, it should be understood that the patient can be any person such as a recipient of service (e.g., a client, customer, participant, or other classification of person receiving service).

In step 3002, a selection of a user role can be received or determined. A user role can include, but is not limited to, a physician, nurse, diagnostic technician, or patient. The user role may include any role in a healthcare setting or other professional environment. The user can select the user role via text input, button, drop-down menu, selection from a list, or other method of selection known in the art. As an example, the user can select the user role using a user device 124 or a client device 102. However, other devices and systems can be used to select the user role. The user role can be determined based upon an identification of the particular user, a user record associated with the user, and/or user settings associated with a user. As an example, the computing device 104 can determine a user role from a preset number of defined user roles based on the user accessing the computing device 104 or system associated therewith.

In step 3004, a user interface (e.g., dashboard) and/or functions associated therewith can be modified according to the selected user role. As an example, the information and functions that are irrelevant, less relevant, or prohibited by the identified role are less prominent, hidden, or altogether absent from the user interface. On the other hand, information and functions that are more important to the role are featured prominently in the user interface. The user interface can also include one or more displays or dashboards customized to a particular role that display the information that is most relevant to the role.

In an example for the nurse role, a nurse is most focused on the patients that are assigned to his or her care during his or her shift. A nurse's responsibilities include making sure that the patients are comfortable; the patients are accounted for; the patients are ready for any procedures, surgeries, lab work, or diagnostics; and the patients are given their medication on time.

Accordingly, in an example, the user interface for the nurse role can be modified to include an option to enable a display or dashboard showing the patients currently under the nurse's care. This allows the nurse to quickly see for which patients in the unit the nurse is responsible. From this display or dashboard of the nurse's patients, the nurse can select a patient and quickly access that patient's full details and record.

As another example of a modification for the nurse role, the user interface can be modified to include an option to enable a display or dashboard showing the schedule for the patients under the nurse's care. The schedule can include, for example, one or more of patients' procedures, surgeries, lab work, or diagnostics. The schedule enables the nurse to keep track of his or her patients and make sure that the patients are ready for upcoming events on the schedule. For example, if a patient is scheduled for surgery, a nurse will be able to see on the nurse's schedule dashboard that the surgery is scheduled and can timely prepare the patient, such as making sure the patient didn't eat or drink before the surgery and are in the proper surgery garments.

As another example of a modification for the nurse role, the user interface can be modified to include an option to enable a display or dashboard showing the medication schedule for the nurse's patients. This allows the nurse to easily see which of her patients need to be given medication and when. This will assist the nurse in making sure that all of the nurse's patients receive their medication and on time.

As another example of a modification for the nurse role, the user interface can be modified to include an option to transfer patients to one or more other nurses. In a hospital or other healthcare facility, a nurse usually works a particular shift. At the end of the nurse's shift, a new shift arrives to relieve the nurse and take over the care of the nurse's patients. The user interface can be modified to facilitate this transfer of patients. The interface can include an option to transfer all of the nurse's patients to the incoming nurse at once. As another example, in the event that the nurse's patients are being split among more than one incoming nurse, the interface can include an option to individually or in a subset transfer patients to an incoming nurse. As another example, the interface can be configured to automatically transfer the outgoing nurse's patients to the incoming nurse or nurses scheduled to take over the outgoing nurse's patients.

As another example of a modification for the nurse role, the user interface can be modified to include an option to send a message to one or more nursing shifts. In this example, a nurse is given an option to send a message to, for instance, the nurse's fellow nurses on the shift or to the nurses on other shifts. This modification serves to facilitate communication both among the nurses on a shift and between shifts.

In an example for the physician role, the physician is responsible for the high-level care of his or her patients. A physician's responsibilities include checking in on patients; ordering diagnostics, lab work, and procedures; prescribing medications; performing procedures; and ordering the discharge of patients.

In an example of a modification for the physician role, the user interface can be modified to include an option to enable a display or dashboard of the patients under the physician's care. This can allow the physician to quickly view all the patients for which he or she is responsible. From this display or dashboard of the physician's patients, the physician can select a patient and quickly access that patient's full details and record.

As another example of a modification for the physician role, the user interface can be modified to include an option to enable a display or dashboard showing a schedule. In an aspect, the schedule can show the diagnostics, lab work, procedures, and surgeries scheduled for the physician's patients. This schedule display or dashboard can allow the physician to see a quick snapshot of his or her patients' events and care. In another aspect, the schedule can show the tasks that are scheduled for the physician. The physician's tasks can include appointments, meetings, patient evaluations, and procedures or surgeries that the physician is to perform. This schedule display or dashboard serves to give the physician an overview of his tasks for a time period.

As another example of a modification for the physician role, the user interface can be modified to include an option to display deficiencies or outstanding tasks that must be completed before another scheduled event can take place. For example, before a surgery can be performed, certain diagnostics or lab work, such as X-rays and blood tests, must be completed and the patient must grant his or her consent. In an exemplary user interface modification, if a physician had ordered a patient to undergo surgery, the option can display to the physician a list of the tasks that must be completed before the surgery can be performed. In this case, the list would include X-rays, blood tests, and a consent agreement. The user interface may further include options to facilitate completion of the deficiencies or outstanding tasks. For example, in the case of an outstanding X-ray, the interface can include options to order the X-ray, schedule the X-ray with the hospital's radiology department, and/or send a communication to the patient's nurse.

As another example of a modification for the physician role, the user interface can be modified to include an option to capture a record of a physician's time. The record of the physician's time can be used to record the time spent with a patient or spent completing a task. For example, the user interface can include an option for the physician to click a button to record the start time of, for instance, a patient consultation and to click a button to record the end time of the consultation. As another example, the user interface can include an option for the physician to manually enter the start and end times of a time period. As another example, the user interface can include an option for the physician to manually enter an elapsed time of a time period. The record of a physician's time can be used for billing or time-planning purposes, for example.

As another example of a modification for the physician role, the user interface can be modified to include functions that are only enabled for physician users. Since the physician is the healthcare provider that is ultimately responsible for the patient's care, certain healthcare tasks can only be ordered or completed by a physician. For example, usually only a physician can order diagnostics, lab work, procedures, and surgeries; prescribe medication; and initiate a patient's discharge. Accordingly, the user interface modified for a physician user can have the aforementioned functions enabled. Conversely, these functions would be disabled or not visible to other user roles, such as a nurse user. This has the additional benefit to the non-physician users since their interface will not be cluttered by functions that are irrelevant to their role.

In an example for the diagnostic technician role, a diagnostic technician is responsible for scheduling diagnostic tests or procedures, performing the diagnostic tests or procedures, and returning the diagnostic test or procedure results back to the ordering physician or other healthcare provider. A diagnostic technician can include, for example, an X-ray technician, CT scan technician, or blood lab technician.

In an example of a modification for the diagnostic technician role, the user interface can be modified to include an option to show a display or dashboard showing the schedule for the diagnostic technician. The schedule can show the upcoming patients scheduled to have a diagnostic test or procedure performed by the diagnostic technician and/or his or her department or unit. The interface may allow the user to select a patient to see more information about the patient, such as height, weight, and medical history, and about the order, such as the name and contact information of the ordering physician.

In an example of a modification for the diagnostic technician role, the user interface can be modified to include an option to communicate with a patient's physician or other healthcare provider associated with the patient. For example, the user interface can include an option to send a message to the ordering physician to clarify an instruction in the order. In addition, the user interface can be modified to include an option to return the diagnostic test or procedure results back to the ordering physician or other healthcare provider.

In an example for the patient role, the patient is generally only concerned about aspects of his or her own care. A patient may like to know who his or her current healthcare providers are. A patient may want to know what medications he or she has been prescribed. He or she may like to know what procedures, diagnostics, or lab work are scheduled and for when they are scheduled. Additionally, a patient may like to review the results of a diagnostic test or lab work.

Accordingly, in an example of a modification for the patient role, the user interface can be modified to include an option to show one or more displays or dashboards. For example, a display or dashboard can include information on a patient's healthcare providers. The display or dashboard can include a text or graphical representation of the patient's physicians and current nurse, for example. If the patient is transferred to another physician or the nursing shift rotates, the display or dashboard is updated accordingly.

As another example, a display or dashboard can include information on the medication, therapies, treatments, and the like that the patient is prescribed. As an illustration, the display or dashboard can show that a patient is given 200 mg of ibuprofen twice a day and is prescribed physical therapy twice a week. The interface may, for example, allow the user to select a medication, therapy, or treatment and receive more information on that medication, therapy, or treatment. For example, a user may be able to select ibuprofen and be shown a description of the usual uses of the medication and the possible side effects. A user may be able to select the physical therapy item on the display or dashboard and be shown a description of what physical therapy entails and the name of the physical therapist.

As another example, a display or dashboard can include information on a patient's schedule. The display or dashboard can include a text or graphical representation of the patient's schedule. Items on the schedule can include medication, a treatment, therapy, a diagnostic test, lab work, a procedure, or a surgery. The interface may allow the user to select an item on the schedule to receive more information about that item. For example, a user can select an X-ray item on the user's schedule and see information about X-rays. A user can select a surgery on the user's schedule and see information on the surgery, such as how it is performed and what to expect during recovery.

As another example, a display or dashboard can include information on a patient's diagnostic data. The display or dashboard can include a text or graphical representation of the patient's diagnostic data. As an illustration, if a patient had an X-ray performed, he or she could select the X-ray diagnostic data representation and see the X-ray image. As another illustration, if a patient had blood work performed, he or she could select the blood work diagnostic representation and view the results of the blood work, such as cholesterol and iron levels.

### Communication Session File

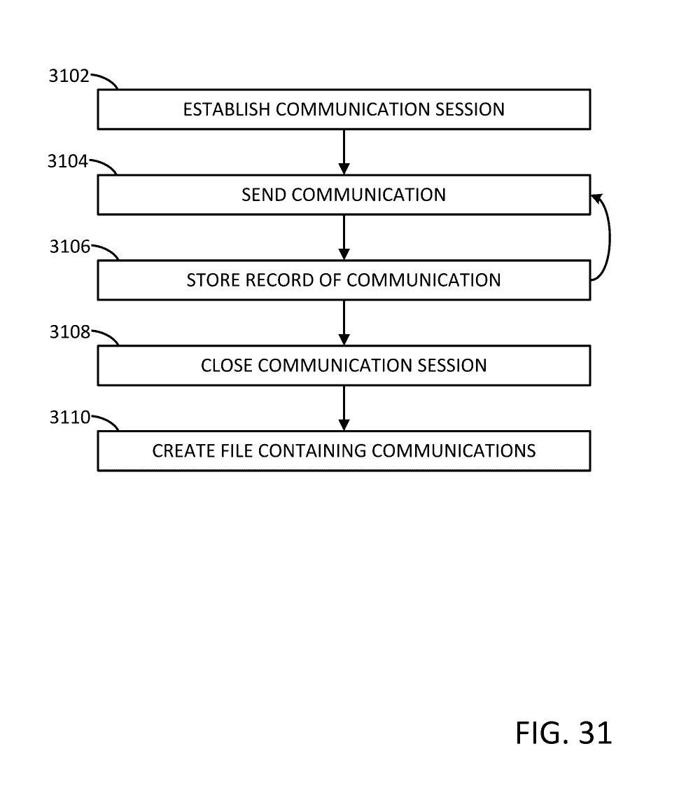

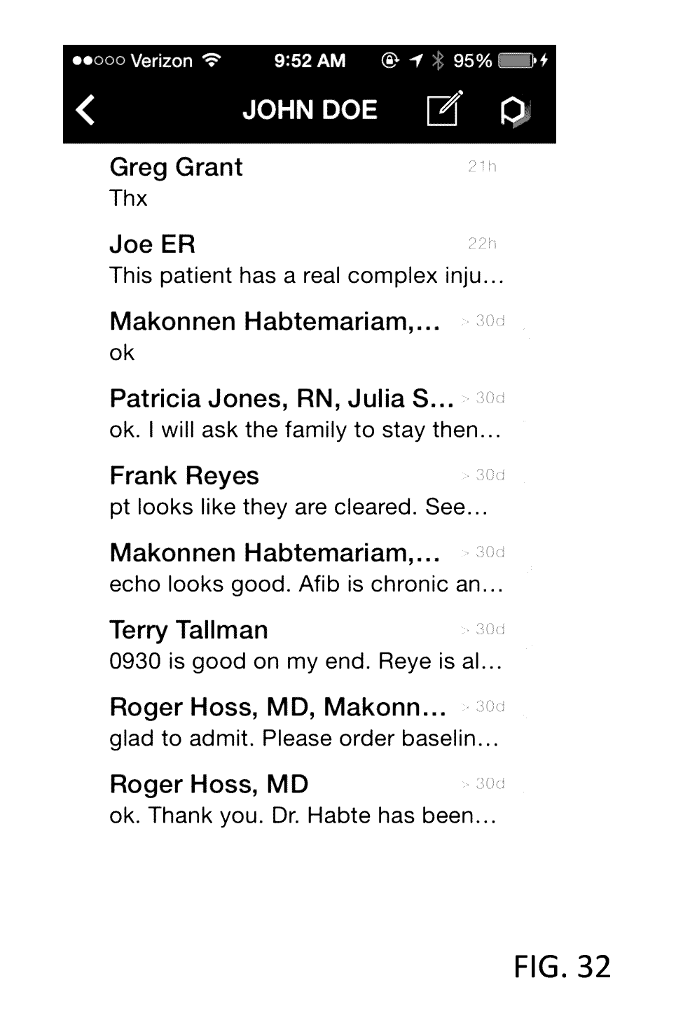

FIG. 31 illustrates an exemplary method for generating a file documenting a communication session between service providers such as healthcare providers. In step 3102, a communication session can be established between two or more participants. In an example, the communication session is associated with a patient (e.g., patient profile) or other individual. A participant of the communication session can include a primary care physician, an emergency department (ED) physician, a hospital physician, a nurse, or a diagnostic technician. As an illustrative example, a communication session can be established when a patient arrives at a hospital ED. In this example, the communication session can be associated with the patient. The participants in the communication session can include an ED physician, the patient's primary care physician, a nurse, and an X-ray technician. Any number of participants can be included in the session, added, or removed during the period in which the communication session is open. As a further example, the communication session can facilitate the exchange of information through one or more secure network connections. In certain aspects, the communication session can comprise a host server making available a patient profile and all updates to the profile provided by one or more users in real-time. As such, any communications (e.g., messages), labs, notes, remarks, and the like provided by one or more participants of the communication session can be shared among all of the participants in the communication session.

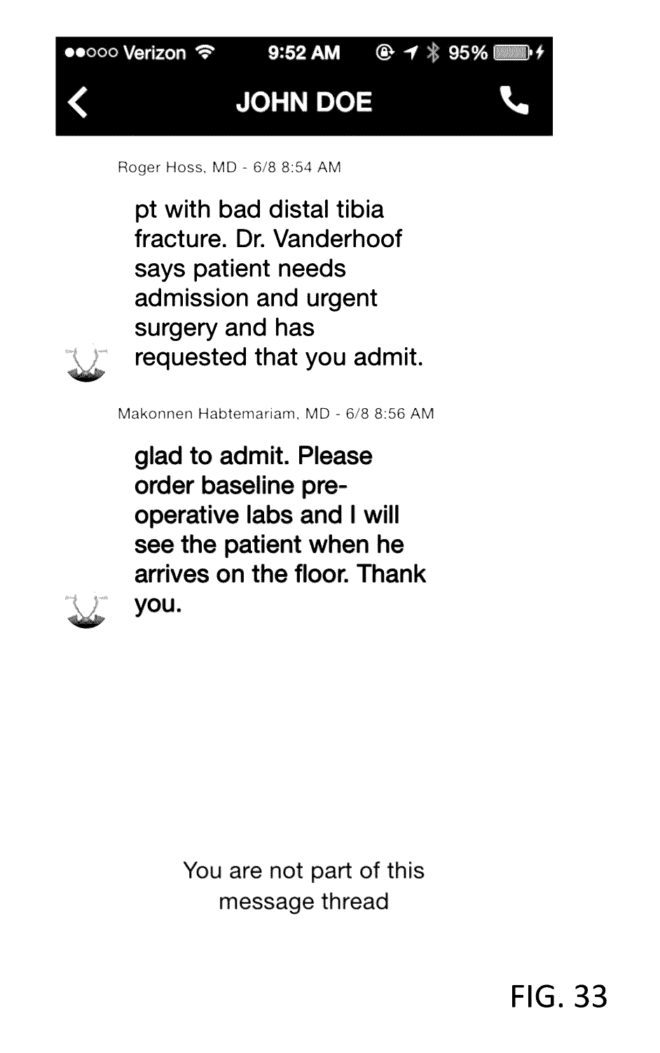

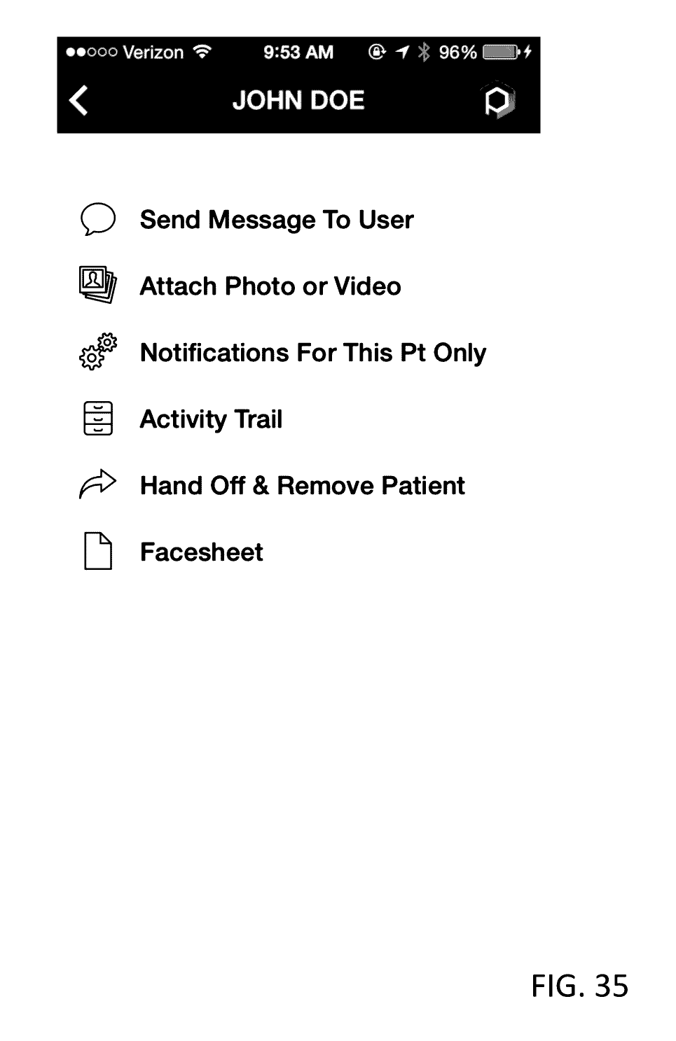

In step 3104, a participant can send a communication to one or more other participants in the communication session. As an example, collaborative messaging is illustrated in FIG. 32. In certain aspects, a non-participant can view a communication session, such as shown in FIG. 33. In certain aspects, one participant may ‘hand off’ the communication session to another user to allow the new user to participate in the session, as shown in FIG. 35. A communication in the communication session can include a secure messaging system facilitating HIPAA-compliant exchanges of information. The communication can also include data, such as lab results or an X-ray image. The method of communication can also include a server-side communication system where the communication is performed via the computing device 104. In this method, a participant can access communications through a client connected to the computing device 104. Such a client can include an application client that runs on a desktop computer operating system (e.g., Windows® or OS X®), an application client that runs on a mobile device operating system (e.g., Android® or iOS®), or a client that runs in a web browser (e.g., via an http protocol).

Continuing the illustrative ED example, after the patient arrives at the ED and a communication session is created and associated with the patient, a healthcare provider participant can send a message to another healthcare provider participant. For example, when the ED physician examines the patient for the first time, the ED physician can have a question about the patient's current medications. The ED physician can send a message to the patient's primary care physician inquiring about what medications the patient is taking. In turn, the primary care physician can respond with a list of the patient's medications. After the ED physician decides to admit the patient to the hospital, the ED physician can send a message to the primary care physician informing the primary care physician of the admittance. Later, an X-ray can be taken of the patient. The X-ray technician can send the X-ray image as a communication to the ED physician and the primary care physician. In an aspect, any user that is linked to the patient profile of the subject patient can be notified of the communications and updates occurring in the communication session.

In step 3106, a record of at least a portion of the communication session can be stored. The record can be stored on any storage medium. As an example, the record can be stored in the database 114 of the computing device 104. In addition, the record can be stored in any format and in any storage medium connected to the computing device 104. The stored record can be associated in the storage system with the communication session. After the record is stored, additional communications can be made and subsequently stored.

Continuing the illustrative ED example above, after a communication by a healthcare provider is performed, the communication is stored. For example, the ED physician's message to the primary care physician regarding the patient's medication and the primary care physician's response can be stored in the system. The ED physician's message documenting the patient's admittance to the hospital can be stored. Finally, the X-ray of the patient can be stored. In an aspect, the communication session may be stored as a record once the session is closed and no further communication are immediately necessary. Such storage can comprise the creation of a formatted file (e.g., PDF) that can be associated with an EMR or records at a PCP's office.

In step 3108, the communication session can be closed. A communication session can be closed after any finite period of time. For example, a communication session can be closed after just a brief dialogue about a patient between two physicians. In another example, the communication session can be closed after a more protracted period, such as after a patient is discharged from a hospital after a one-month stay.

Continuing the illustrative ED example above, instead of being admitted to the hospital, the ED physician treats the patient in the ED and discharges the patient. When the patient is discharged, the communication session associated with the patient can be closed. As a further example, the communication session may remain open until a follow-up appointment with the PCP is scheduled. Other circumstances can be used to close a communication session.

In step 3110, a file is created that contains all of the communications of the communication session. In an embodiment, the format of the file is portable document format (pdf). The file can include, in addition to the communications, ancillary information to form an audit trail of the communication session. The ancillary information can include timestamps for when a communication took place, the communication creator, and the communication recipient(s). The file can be sent to one or more systems associated with the participants or the patient, such as an office system 125 or information system 108.

Continuing the illustrative ED example above, after the patient's communication session is closed, a pdf file is created that includes the ED physician's message to the primary care physician, the primary care physician's response, the ED physician's admittance email, and the X-ray image. The pdf file can then be sent to the office system 125 of the primary care physician so that the communications documenting the patient's ED visit can be added to the patient's file in the primary care physician's office system 125. In addition, the pdf file can be sent to the ED hospital's information system 108 to supplement the hospital's records of the visit.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications are referenced. The disclosures of these publications in their entireties are hereby incorporated by reference into this application in order to more fully describe the state of the art to which the methods and systems pertain.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A computer-implemented method for providing patient-centric management of a patient service and information relating to the patient service, wherein the patient service includes a plurality of medical professionals, the method comprising:
generating, by one or more processors, a patient profile comprising a patient identifier and information relating to the patient identifier;
linking one or more medical professionals of the plurality of medical professionals to the generated patient profile, wherein the one or more linked medical professionals are capable of accessing the patient profile over a secure network connection and communicating with each other over the secure network connection;
receiving, by the one or more processors, a message relating to the patient profile;
automatically transmitting, by the one or more processors, a notice to the one or more linked service providers, wherein the notice indicates that a message has been received relating to the patient profile;
receiving, by the one or more processors, a request to access the patient profile from the one or more linked service providers in response to the notice; and
granting access to the patient profile including the update, via the secure network connection.

2. The method of claim 1, wherein the patient profile comprises medical information relating to a particular patient.

3. The method of claim 2, wherein the medical information is compiled from at least two discrete healthcare systems.

4. The method of claim 1, wherein generating the patient profile comprises receiving medical information and sorting the medical information based upon medical relevance.

5. The method of claim 1, wherein generating the patient profile comprises receiving medical information and including only a subset of the received medical information in the patient profile, wherein the subset is based on a medical relevance.

6. The method of claim 1, wherein the granting access to the patient profile comprises transmitting medically relevant information to one of the linked medical professionals.

7. The method of claim 1, wherein the granting access comprises providing credentials to an unlinked service provider.

8. The method of claim 1, further comprising storing communications between the linked service providers as part of the patient profile.

9. A computer-implemented method for providing patient-centric management of a patient service and information relating to the patient service, wherein the patient service includes a plurality of service providers, the method comprising:
generating, by one or more processors, a patient profile comprising a patient identifier and information relating to the patient identifier;
linking one or more service providers of the plurality of service providers to the generated patient profile, wherein the one or more linked service providers are capable of accessing the patient profile over a secure network connection;
receiving, by the one or more processors, an update relating to the patient profile;
automatically transmitting, by the one or more processors, a notice to the one or more linked service providers, wherein the notice indicates that an update has been received relating to the patient profile;
receiving, by the one or more processors, a request to access the patient profile from the one or more linked service providers; and
granting access to the patient profile including the update, via a secure network connection.

10. The method of claim 9, wherein the patient profile comprises medical information relating to a particular patient.

11. The method of claim 10, wherein the medical information is compiled from at least two discrete healthcare systems.

12. The method of claim 9, wherein generating the patient profile comprises receiving medical information and sorting the medical information based upon medical relevance.

13. The method of claim 9, wherein generating the patient profile comprises receiving medical information and including only a subset of the received medical information in the patient profile, wherein the subset is based on a medical relevance.

14. The method of claim 13, wherein the medical information is compiled from at least two discrete healthcare systems.

15. The method of claim 9, wherein the update to the patient profile comprises receiving a message relating to the patient profile.

16. The method of claim 15, wherein the message is received from one of the linked service providers.

17. The method of claim 9, wherein the granting access to the patient profile comprises allowing a user to transmit a message to or receive a message from other users, wherein the message relates to the patient profile.

18. The method of claim 9, wherein the granting access to the patient profile comprises transmitting medically relevant information to a user.

19. The method of claim 9, wherein the granting access comprises providing credentials to an unlinked service provider.

20. The method of claim 9, further comprising storing communications between the linked service providers as part of the patient profile.
